(19) United States
(12) Patent Application Publication
(10) Pub. No.: US 2011/0301091 A1
(43) Pub. Date: Dec. 8, 2011

(54) COMPOSITIONS AND METHODS FOR REDUCING THE SIGNS OF AGING OF THE SKIN

(76) Inventors: Giammaria Giuliani, Milan (IT); Raymond Rodriguez, Davis, CA (US); Kevin Dawson, Davis, CA (US)

(21) Appl. No.: 13/127,249

(22) PCT Filed: Nov. 3, 2009
(86) PCT No.: PCT/US2009/063137

§ 371 Date: Jul. 7, 2011

Related U.S. Application Data
(60) Provisional application No. 61/198,235, filed on Nov. 3, 2008.

(51) Int. Cl. A61K 38/16; A61Q 19/08; A61P 3/02; A61P 17/00; A61K 38/02; C12Q 1/02

(52) U.S. Cl. 514/18.8; 514/18.6; 435/29

(57) ABSTRACT

Compositions and methods used to reduce the visible signs of aging of the skin by recalibrating the expression of genes, genetic networks, and cellular pathways in the human skin, particularly using combinations of natural compounds that produce synergistic effects on the expression of genes and genetic networks.

### RELATIONSHIP TO OTHER APPLICATIONS

This application claims the benefit of and priority to U.S. Provisional application No. 61/198,235 filed 3 Nov. 2008 and titled “Compositions and methods for reducing the signs of aging of the skin”.

### FIELD OF THE INVENTION

The invention relates to compositions and methods used to reduce the visible signs of aging of the skin, particularly to the prevention and reduction of skin wrinkles and to the production and maintenance of youthful looking skin. The compositions and methods employ compounds that affect the expression of various genes within the skin which genes are involved in chronological-induced and UV-induced skin damage. The invention relates to methods for recalibrating the expression of genes, genetic networks, and cellular pathways in the human skin, primarily in the dermis, that have changed as a result of the chronological aging process. The invention also relates to combinations of natural compounds that produce synergistic effects on the expression of genes relevant to the reversal of skin aging and skin cancer risk reduction.

### BACKGROUND

The skin is the largest human organ. It covers between 1.5 and 2 m², comprising about one sixth of total body weight. The skin performs a complex role in human physiology: serves as a barrier to the environment, and the sebum produced by some of its glands (sebaceous) have anti-infective properties. The skin acts as a channel for communication to the outside world, protects us from water loss, friction wounds, and impact wounds and uses specialized pigment cells to protect us from ultraviolet rays of the sun. Skin produces vitamin D in the epidermal layer, when it is exposed to the sun's rays. The skin helps regulate body temperature through sweat glands and helps regulate metabolism. The skin consists of three functional layers: Epidermis, the Dermis (or corium) and the Subcutis (or hypodermis).

Various cell types are present in the skin. Keratinocytes is the most abundant cell type in the epidermis. These cells produce keratin proteins. Fibroblasts differentiate into cells that form the dermis and produce collagen and elastin. Melanocytes produce the pigment melanin that accumulates around the nuclei of the keratinocytes absorbing harmful ultraviolet (UV) light. Langerhans cells (macrophages) reside in the dermis mediating humoral and cellular immune functions. Merkel's cells, which are present in small numbers but are more numerous in the skin of the palms and soles of the feet, are sensory mechanical receptors that respond to certain stimuli such as pressure or touch.

The epidermis is the outermost skin layer. As skin cells migrate to the surface, farther away from their source of nourishment, they flatten and shrink. They lose their nuclei, move out of the basal layer to the horny layer (stratum corneum), and die. This process, called keratinization, takes about 4 weeks. About 10 percent of epidermal cells are melanocytes that pigment the skin. The epidermis is differentiated into five layers: horny layer (stratum corneum); clear layer (stratum lucidum); granular layer (stratum granulosum); prickle-cell layer (stratum spinosum); and the basal layer (stratum basale).

The dermis is the layer just below the outer keratinized epidermal layer. The dermis contains cells, water, collagen fibers, glycosaminoglycans and fibronectins that form a hydrated gel and are responsible for the high elasticity and tensile strength of the dermis. Embedded in this layer are lymph channels, blood vessels, nerve fibers, muscle cells, hair follicles, sebaceous glands, and sweat glands.

Glycosaminoglycans are mucopolysaccharides present in the dermis that can bind large amounts of water. As the skin ages, the interweaving of the collagen fibers increases and the water-binding capacity diminishes and the skin tends to wrinkle. Glycosaminoglycans bind with the proteins in the connective tissue matrix to form proteoglycans. These proteoglycans form a gel-like material that can absorb and expel water like a sponge. Glycosaminoglycans are subject to a continuous turnover. In contrast, the collagen fibers are only renewed when necessary, such as when injury is sustained.

The ability of the skin to store water and thereby remain soft and supple depends in part on the presence of lipids, arginine, and other “natural moisturizing factors” (NMF) that originate from the cornification (differentiation) of the keratinocytes, for example, pyrrolidine carboxylic acid, and secretions from the sweat and sebaceous glands including urea, salts, and organic acids.

The dermis also contains collagens. Type I collagen is the most abundant protein in skin connective tissue, which also contains other types of collagen (III, V, VII), elastin, proteoglycans, fibronectin, and other extracellular matrix proteins. Newly synthesized type I procollagen is secreted into the dermal extracellular space where it undergoes enzymatic-processing, arranging itself into a triple helix configuration. The triple helix complexes associate with other extracellular matrix proteins such as leucine-rich small proteoglycans, to form regularly arranged fibrillar structures. This process, called fibrillogenesis, results in formation of collagen bundles that are responsible for the strength and resiliency of the skin.

Skin aging is influenced by several factors, including genetics, environmental exposure (ultraviolet (UV) irradiation, xenobiotics, and mechanical stress), hormonal changes, and metabolic processes (generation of reactive chemical compounds such as activated oxygen species, sugars, and aldehydes). Taken together, these factors lead to cumulative alterations of skin structure, function, and appearance. The influence of the environment, especially solar UV irradiation, is of considerable importance for skin aging. Skin aging due to UV exposure (photoaging) is superimposed on chronological skin aging. Historically, scientists considered photoaging and chronological skin aging as two distinct entities. Although the typical appearance of photoaged and chronologically aged human skin can be readily distinguished, recent evidence indicates that chronologically aged and UV-irradiated skin share important molecular features including altered signal transduction pathways that promote matrix-metalloproteinase (MMP) expression, decreased procollagen synthesis, and connective tissue damage. This concordance of molecular mechanisms suggests that UV irradiation accelerates many key aspects of the chronological aging process in human skin. Based on this relationship between UV irradiation and chronological aging, acute UV irradiation of human skin may serve as a useful model to study molecular mechanism of skin chronological aging.

At the tissue level, chronologically aged skin shows general atrophy of the extracellular matrix reflected by decreased number of fibroblasts, and reduced levels of collagen and elastin. The organization of collagen fibrils and elastin fibers is also impaired. This impairment is thought to result from both decreased protein synthesis that particularly affects types I and III collagens in the dermis and increased breakdown of extracellular matrix proteins.

Photodamaged skin is associated with increased epidermal thickness and alterations of connective tissue organization. The hallmark of photoaged skin is accumulation of amorphous elastin-containing material that resides beneath the epidermal dermal junction. Impairment of the fibrillar organization of collagen and elastin is typically more severe in photoaged skin, compared to sun-protected chronologically aged skin. The severity of photoaging is proportional to accumulated sun exposure and inversely related to the degree of skin pigmentation. Individuals with fair skin are more susceptible to solar UV-induced skin damage than darker-skinned individuals.

At the cellular level, one of the earliest detectable responses of human skin cells to UV irradiation is activation of multiple cytokine and growth factor cell surface receptors, including epidermal growth factor receptor (EGF-R), tumor necrosis factor (TNF) alpha receptor, platelet activating factor (PAF) receptor, insulin receptor, interleukin (IL)-1 receptor, and platelet-derived growth factor (PDGF) receptor.

Activation of cell surface cytokine and growth factor receptors results in recruitment in cytoplasm of adaptor proteins that mediate downstream signaling. Assembly of these signaling complexes results in activation of small GTP-binding protein family members which are key upstream regulators of the certain MAP kinases. The action of certain GTP-binding proteins results in an increased formation of superoxide anions. This increased production of ROS likely participates in amplification of the signal leading to the activation of the downstream enzyme complexes such as MAP kinase. ROS are necessary participants in multiple MAP kinase pathways.

Increased intracellular ceramide content may also contribute to activation of the MAP kinase pathways by UV irradiation. UV-induced ceramide generation seems to be dependent on increased ROS production, since ceramide and ROS levels rise in parallel, and UV-induced ceramide production is inhibited by the free radical scavenger Vitamin E.

Now the UV-induced signal cascades enter the nucleus. MAP kinase activation results in induction of transcription factor AP-1 that is a major effector of the MAP kinase pathways. AP-1 regulates expression of many genes involved in the regulation of cellular growth and differentiation. Transcription of several MMP (matrix-metalloproteinase) family members is strongly regulated by AP-1. Several MMPs are upregulated by AP-1. These include MMP-1 (interstitial collagenase or collagenase 1) which initiates degradation of types I and III fibrillar collagens, MMP-9 (gelatinase B), which further degrades collagen fragments generated by collagenases, and MMP-3 (stromelysin 1), which degrades type IV collagen of the basement membrane and activates pro-MMP-1.

MMP induction is, in part, responsible for UV-induced damage to skin connective tissue. Together, MMP-1, MMP-3, and MMP-9 have the capacity to completely degrade mature fibrillar collagen in skin. Consistent with this, increased collagen breakdown has been demonstrated within 24 h after UV irradiation in human skin in vivo. Thus, UV irradiation of human skin causes extracellular matrix degradation via induction of transcription factor AP-1 and subsequent increased MMP production.

In addition to causing collagen breakdown, UV irradiation impairs new type I collagen synthesis. UV irradiation has been shown to decrease collagen production and impair organization of collagen fibrils in skin in vivo. In addition, increased breakdown of extracellular matrix proteins is also observed in UV-irradiated fibroblasts in vitro and in human skin in vivo. Down-regulation of type I collagen is mediated in part by UV-induced AP-1, which negatively regulates transcription of both genes that encode for type I procollagen (COL1A1 and COL1A2).

UV-induced down-regulation of collagen synthesis also occurs via paracrine mechanisms involving transforming growth factor-beta (TGF-beta) and other cytokines. TGF-beta is a major profibrotic cytokine, which regulates multiple cellular functions including differentiation, proliferation, and induction of synthesis of extracellular matrix proteins. The biological effects of TGF-beta are diverse and strongly dependent on its expression pattern and cell type. In human skin, TGF-beta inhibits growth of epidermal keratinocytes and stimulates growth of dermal fibroblasts. Moreover, TGF-beta induces synthesis and secretion of the major extracellular matrix proteins collagen and elastin. TGF-beta also inhibits expression of certain specific enzymes involved in the breakdown of collagen, including MMP-1 and MMP-3. TGF- also has the ability to affect gene expression by epigenetic modification of DNA. Exogenous TGF-beta was shown to induce and maintain expression of Foxp3 in regulatory T cells by demethylating a highly conserved region of the Foxp3 gene called Treg-specific demethylation region (TSDR) [J. K. Polansky et al., 2008. Eur. J. Immunol. 38: 1654-1663]. Both aging and UV irradiation induce molecular alterations that create skin aging. A major feature of aged skin is the reduction of types I and III procollagen synthesis. This reduction results in skin thinning and increased fragility. Both types I and III procollagen mRNA and protein expression are reduced in aged skin.

In addition to impaired collagen synthesis, increased production of several MMP family members, including MMP-1, MMP-2 (gelatinase A), MMP-3, and MMP-9 occurs in chronologically aged skin. With the exception of MMP-2, these MMPs are regulated by AP-1 and induced by UV irradiation. Interestingly, AP-1 expression is increased in aged human skin in vivo and aged skin fibroblasts in vitro.

Oxidative stress is thought to be of primary importance in driving the aging process. The free radical theory of aging, first proposed several decades ago, envisions that the molecular basis of aging derives from accumulation, over a lifetime, of oxidative damage to cells resulting from excess ROS, which are produced as a consequence of aerobic metabolism. Although skin possesses extremely efficient anti-oxidant activities, it has been demonstrated that during aging, ROS levels rise and anti-oxidant defenses decline. ROS are necessary participants in multiple MAP kinase pathways. MAPK activation results in induction of AP-1, which in turn, upregulates expression of MMPs. This scenario provides a plausible mechanism for the observed increased collagen degradation in aged human skin.

In spite of existing differences, many critical molecular features of aged and UV-irradiated human skin bear striking similarities. It could be stated that these similarities reflect the central role that oxidative stress plays in UV irradiation-induced responses and aging in human skin. Viewed in this light, it is not surprising that UV irradiation and aging evoke similar molecular responses, since both are responding to oxidative stress. Nor is it surprising that the consequences of UV irradiation and aging have similar damaging impact on skin connective tissue.

### BRIEF DESCRIPTION OF THE INVENTION

This disclosure describes a method for reversing signs of skin aging and risk of skin cancer by recalibrating the expression of genes, genetic networks, and cellular pathways in the human skin, primarily in the dermis, that have changed as a result of the chronological aging process. Gene expression patterns, and the pathways they participate in, are restored to levels characteristic of a younger chronological age by treating the skin with specific combinations of natural compounds (e.g., phyto-chemicals, nutrients, minerals, vitamins, etc). Specific combinations of natural compounds are determined using informatic algorithms and high throughput screening. Natural compounds are delivered to the dermis topically with dermo-cosmetics and internally with oral supplements. Combinations of natural compounds are claimed that produce synergistic effects on the expression of genes relevant to the reversal of skin aging and skin cancer risk reduction. Natural compounds can affect gene expression directly (e.g., transcription factor agonists or antagonist) or indirectly (e.g., non-coding RNAs, epigenetic modifications, signaling receptor agonists or antagonist). The invention includes those natural compounds that produce synergistic effects on gene expression when administered both orally and topically. Also disclosed are those genes, gene networks, noncoding RNAs and epigenetic modifications associated with chronologically younger or older skin.

The invention encompasses the following embodiments which are merely exemplary embodiments, not meant to limit the invention.

The methods described may be used to discover new chemical entities and combinations of chemical entities, natural and man-made, that will alter aging-related gene expression profiles and signatures.

1. A method for reversing signs of skin aging and risk of skin cancer by recalibrating the expression of genes, genetic networks, and cellular pathways in the human skin, primarily in the dermis, that have changed as a result of the chronological aging process wherein the genes are selected form the group consisting of specific genes are listed in Table 1, genetic networks, cellular pathways, and other functional categories are listed in Table 3.

2. The method of the above described embodiment No. 1 wherein the genes recalibrated comprise one or more genes from Table 1 or genes selected from the group consisting of the genes of the functional categories listed in Table 3.

3. The method of the above described embodiment No. 1 wherein the genetic networks or cellular pathways recalibrated comprise one or more selected from the groups consisting of the genetic networks or cellular pathways listed in Table 3.

4. A method for reducing the signs of aging of the skin the method comprising applying to the skin a compound that was identified as having recalibrating potency with the method of the above described embodiment No. 1.

5. The method of the above described embodiment No. 4 wherein the compound comprises a chromatin remodeling protein or peptide for example lunasin or a variant or derivative thereof.

6. A method for reducing the signs of aging of the skin the method comprising topically applying to the skin a compound “A” having anti-aging properties identified in the above described embodiment No. 1 and further comprising orally administering a compound “B” having anti-aging properties identified in the above described embodiment No. 1.

7. The method of the above described embodiment No. 6 wherein compounds A and B, when administered contemporaneously, provide a synergistic effects on expression of genes of Table 1 or groups of Table 3.

8. A method for amplifying expression of genes, which genes are normally activated by hormones, drugs, extracellular and other environmental stimuli, the method comprising administering to a subject a therapeutic amount of chromatin remodeling protein/peptide such as lunasin or a variant or derivative thereof thereby increasing chromatin accessibility in the promoter region of the gene due to acetylation of lysine 16 at the amino-terminal ends of histone H4 of nucleosome.

9. A composition for reducing the signs of aging of the skin, the composition comprising one or more substances that alter the expression of genes involved in the biosynthesis or degradation of a substance selected from the group consisting of collagens and elastin, glycosaminoglycans and fibronectins and metallopeptidases.

10. The composition of the above described embodiment No. 4 wherein the composition comprises a chromatin remodeling protein or peptide, for example, lunasin or a derivative or variant thereof.

11. A composition for reducing the signs of aging of the skin, the composition comprising one or more substances that increase the expression of genes involved in the biosynthesis of type I or type II collagen.

12. A composition for reducing the signs of aging of the skin, the composition comprising one or more substances that reduce the enzymatic activity of collagenase.

13. A composition for reducing the signs of aging of the skin, the composition comprising one or more substances that reduce the enzymatic activity of enzymes that degrade fibrin or glycosaminoglycans, or a composition comprising one or more substances that increase the production or maintenance of glycosaminoglycans or proteoglycans.

14. A composition for reducing the signs of aging of the skin, the composition comprising one or more substances that reduce the enzymatic activity of MMP enzymes (matrix-metalloproteinases) that degrade collagen.

15. A composition for reducing the signs of aging of the skin, the composition comprising one or more substances that increase type I and III procollagen synthesis.

16. A composition for reducing the signs of aging of the skin, the composition comprising one or more substances that decrease production of MMPs.

17. A composition for reducing the signs of aging of the skin, the composition comprising one or more substances that decrease production of compounds selected from the group consisting of MMP-1, MMP-2 (gelatinase A), MMP-3, and MMP-9.

18. A composition for reducing the signs of aging of the skin, the composition comprising one or more substances that reduce the rate of degradation of the extracellular matrix proteins in the dermis.

19. A composition for reducing the signs of aging of the skin, the composition comprising one or more substances that maintain or increase the number of fibroblasts present in the dermis.

20. A composition for reducing the signs of aging of the skin, the composition comprising one or more substances that maintain or increase the number of collagen fibrils or elastin fibers in the dermis.

21. A composition for reducing the signs of aging of the skin, the composition comprising one or more substances that maintain or increase the number of collagen fibrils or elastin fibers in the dermis.

22. A composition for reducing the signs of aging of the skin, the composition comprising one or more substances that maintain or increase the 3-dimensional extracellular matrix structure of collagen, elastin, and other extracellular matrix proteins in the dermis,

23. A composition for reducing the signs of aging of the skin the composition comprising combinations of natural compounds including phytochemicals, nutrients, minerals, vitamins, etc.

The invention also encompasses compositions of natural compounds, for external application to the skin, that reduce, delay, and/or reverse the signs of aging of the skin; composition of natural compounds, for internal application that reduce, delay, and/or reverse the signs of aging of the skin; compositions of natural compounds that produce synergistic effects on the expression of genes and/or gene products relevant to the reversal of skin aging and skin cancer risk reduction; compositions of natural compounds that affect and/or recalibrate the expression of various sets of genes, genetic networks, and/or cellular pathways in the human skin with the effect of reducing, delaying, and/or reversing the signs of aging of the skin; methods for reducing, delaying, and/or reversing the signs of aging of the skin by the external application and internal administration of claimed compounds; and methods for making above compounds and formulations; and methods for evaluating the efficacy of claimed compounds and formulations.

Description of the Tables

Table 1 is a table of age-related genes. Each mRNA in Table 1 was tested for interaction with microRNAs listed in the miRBase database.
Table 2 is a table of aging-related microRNAs ranked in the decreasing order of the number of interacting mRNAs.
Table 3 shows gene ontology categories and functional categories of proteins that were over-represented in sets of genes having altered expression levels in aged skin.

### DETAILED DESCRIPTION OF THE INVENTION

### Definitions

The term “signs of skin aging” refers to any anatomical visible indication that is generally associated with skin as a person gets older, including wrinkles, sagging, discoloration and reduced suppleness.

The term “recalibrating” when applied to the expression of genes, genetic networks, and cellular pathways refers to a change of adjustment of expression of one or more genes to produce a verisimilitude of a former state, such as the adjustment of expression of one or more genes listed in Table 1 so as to increase the production of glycosaminoglycans, proteoglycans, collagen etc.

The term “genetic network” or “genetic pathway” refers to two or more genes the expression of which is coordinated or related to a single physiological function such as the production of a particular protein or glycosaminoglycan.

The term “variant or derivative” when used in conjunction with a species such as a drug or other chemical entity is used to mean said drug or other chemical entity comprising at least one chemical modification, such as, but not limited to, a moiety, a radical group, a reactive group, a charged group, an uncharged group, an ion, or the like. The chemical modification can be either addition or removal of such moiety, group, ion, or the like.

The term “drug” is used to mean any molecule that alters the physiology of an organism.

The term “protein” includes peptides.

The term “environmental stimulus” is used to mean any stimulus that in some way affects the physiology of an organism and that has its origins outside of the organism.

The term “a therapeutic amount” is used to mean an amount (of a substance) that produces a measurable effect related to the health of an organism.

The term “gene expression” is used to refer to the transcription of a gene or a part of a gene and is independent from translation. The expression of the gene or part thereof can be increased or it can be decreased. Translation of the expressed gene or part thereof can be increased or it can be decreased.

General Representations Concerning the Disclosure

In this specification where reference is made to particular features of the invention it is to be understood that the disclosure of the invention in this specification includes all appropriate combinations of such particular features. The embodiments disclosed in this specification are exemplary and do not limit the invention. As used in this specification, the singular forms “a”, “an”, and “the” include plural reference unless the context clearly dictates otherwise. The term “comprises” and grammatical equivalents thereof are used in this specification to mean that, in addition to the features specifically identified, other features are optionally present. The term “at least” followed by a number is used herein to denote the start of a range beginning with that number. Where reference is made in this specification to a method comprising two or more defined steps, the defined steps can be carried out in any order or simultaneously.

This specification incorporates by reference all documents referred to herein and all documents filed concurrently with this specification or filed previously in connection with this application, including but not limited to such documents which are open to public inspection with this specification.

### DESCRIPTION OF THE INVENTION

This invention relates to skin aging and describes a discovery platform for selecting active ingredients for topical and internal applications and combinations thereof that can slow down the human skin's aging process and/or diminish the visible signs of skin aging.

The skin protects the body against external injuries such as mechanical, chemical, radiation (for example, UV light), and biological (for example, pathogens) injuries and protects the organs and biological fluids in the human body. The skin also plays a role in thermoregulation by changing blood circulation, body hair, and perspiration. The skin is also a sensory organ collecting tactile, vibration, pressure, heat, cold, and pain stimuli from the environment. The skin is also a metabolic organ. UV-irradiated skin produces vitamin D that is converted into calcitriol, a hormone regulating calcium-homeostasis. The skin is also an endocrine organ; it is not only a target for but also a place of sex hormone synthesis. All these organ functions are affected by the aging process of the skin.

Histologically, the skin is divided into three layers, the epidermis, dermis, and subcutaneous layer. The main barrier layer in the skin is the stratum corneum that is the outermost layer of the epidermis. The stratum corneum contains large amounts of keratin protein. The innermost layer of the epidermis, the stratum basale or stratum germinativum contains the self-renewing and tightly connected keratinocytes lying on a basement membrane. After division, these keratinocytes migrate up to the surface of the skin, flatten out, and form the other layers of the epidermis (Stratum spinosum, granulosum, lucidum, and corneum). The epidermis also contains the melanocytes that produce the melanin pigment of the skin.

Below the basement membrane is located the dermis that contains the connective tissue of the skin, the hair follicles, sweat glands, sebaceous glands, apocrine glands, blood vessels, and many nerve endings. The major proteins in the dermis, the collagen and elastin, are produced by the fibroblasts. The composition of the extracellular matrix (ECM) and the dermal-epidermal junction (DEJ), including glycosaminoglycans and heparan sulfate proteoglycans, drastically change during aging, which makes the dermis a potentially good target for dermocosmetics that aim to reduce the signs of aging. Loss of elastic fibers is one of the main attributes to skin aging. The composition and structure of the ECM change during aging. Laser scanning tomography methods have been used to study the organization of the ESC at the morphological level. In addition to its connective tissue function, the dermis is also responsible for delivering nutrients to the cells in the epidermis. The subcutaneous layer of the skin is mainly composed of adipose tissue.

The skin can adapt to changes in body size and motion while maintaining its integrity. It can move flexibly over other organs. In some locations, such as on the palms and soles, the skin is more fixed to the tissues below it. The skin's mobility requires wrinkling of the skin that flattens out when the skin is stretched out over larger areas. As the body ages, the skin loses much of its flexibility. At the same time, the muscles and bones below the skin may be retracting, which in combination lead to the more permanent wrinkles and other signs of skin aging. As the skin ages, its epidermis, particularly the stratum granulosum loses water content. In addition to wrinkles, the aged skin may exhibit dyspigmentation (hyper- and hypopigmentation), teleangiectasia, laxity, roughness, and a sallow appearance and a higher risk to skin cancers and infections and a lower resistance against mechanical injuries and slower wound healing Cosmetic signs of skin aging include the appearance of lines, wrinkles, age spots, unevenness of tone, and dark circles, skin atrophy and dryness.

In a social context, age is an important external indicator of personal and professional health, well-being, attractiveness, and value. Although cultural variations exist in how older age is valued, in most western societies growing older is perceived as a negative attribute. Therefore, many people, both men and women, seek products and treatments that can restore the youthful appearance of the human skin and prevent the signs of skin aging. Because people in western societies live and work longer, there is a growing demand for anti-aging skin products and treatments. This is reflected in the increased interest in aesthetic and cosmetic dermatology in recent years.

Not every individual's skin change with the chronological age in the same way. Genetic and environmental factors affect when and to what degree certain signs of aging appear. Skin type, pigmentation, diet, UV exposure, infra-red exposure, smoking status, chronic inflammation, exposure to certain chemicals, presence of cellulite, and hormonal status accelerate or retard the skin's aging process. UV-induced aging is particularly well studied, due to its distinct phenotype. This phenotype includes both coarse and fine wrinkles as well as solar elastosis, a condition associated with large accumulations of disorganized elastin in the upper and middle dermis. This type of photo-aging is easy to simulate under laboratory conditions using standard UV irradiation dosimetry studies. It is customary to separate the intrinsic and extrinsic causes of aging and link them to different cutaneuos aging phenotypes.

While intrinsic aging is characterized by atrophy of the dermis due to collagen loss, degeneration of the collagen fiber network, and tissue dehydration, the term extrinsic aging is often used interchangeably with photo-aging. One of the hallmarks of photo-aging is the accumulation of various types of DNA damages in the skin cells and the characteristic solar elastosis. Fluorescent activated cell sorting (FACS) experiments showed that the number of keratinocyte stem cells (KSC) decreases in photoaged skin. These findings suggest that the epidermis of photoaged skin is impaired in terms of its proliferative potential by attempting to repair chronic UV exposure. In addition to collagen another dermal protein, elastin, has pivotal role in maintaining skin elasticity and preventing wrinkles. While collagen synthesis continues throughout an individual's lifetime, the synthesis of skin elastin ceases in young adulthood. Elastase, an elastin degrading enzyme is involved in wrinkle formation of chronological and photoaging. In this process, cytokine expression triggers dermal fibroblasts to increase elastase expression. The increase in elastase activity results in the deterioration of the three-dimensional architecture of elastic fibers, reducing skin elasticity, and finally leading to the wrinkles formation. Although collagen synthesis is maintained throughout life, the collagen matrix in the dermis becomes more and more fragmented with advancing age.

Many topical and systemic treatments have been found effective for the restoration of the photo-aged dermal matrix including all-trans retinoic acid (ATRA), retinyl palmitate, other retinoids, lactose and glycolic acid, lipopentapeptide, lupin peptides, and anti-oxidants. Antioxidants include vitamin C and E, coenzyme Q10, alpha-lipoic acid, glutathione, and others. In animal studies, growth hormone and melatonin was found to slow down dermal aging by a mechanism of reducing oxidative stress and apoptosis. Growth factors and cytokines applied topically were also found beneficial in skin rejuvenation. Sex hormone therapy of skin aging is currently under clinical investigation in both men and women. Dehydroepiandrosterone (DHEA) showed beneficial effects on skin characteristics. In an organotypical model of corticosteroid atrophic human skin, characterized by a decrease of proteoglycans (PG) expression, treatment with C-Xyloside improved expression of heparan-sulfate PGs (HS-PGs) [40]. Topical application of K6PC-5, a recently synthesized novel sphingosine kinase (SK) activator, benefited photoaged skin by improving skin barrier and increasing fibroblast count and function. Carvacol, with its anti-wrinkle effect through the induction of collagen production, might be useful as a possible therapeutic agent or adjunctive agent to retinoic acid for the treatment of skin aging [43 Kim, Ha, Kim, Park, 2008]. Topical application of folic acid and creatine also improved photoaging. Many natural products have been found beneficial in preventing or delaying the signs of skin aging. A fermentable metabolite from Zymomonas mobilis, a Gram-negative facultative anaerobic bacterium, was found to suppress UV-induced wrinkle formation by inhibiting type I procollagen synthesis reduction. Panduratin A, a Kaempferia pandurata Roxb isolate increased type I procollagen synthesis and decreased matrix metalloproteinase (MMP) I synthesis in UV-irradiated human fibroblasts by inhibiting UV-induced activation of mitogen activated protein kinases (MAPKs). Phytoecdysteroids isolated from the seeds of Chenopodium quinoa might be considered as potent chemical agents to prevent or delay both collagenase-related skin damages and oxidative stress. Baicalin, a Chinese herbal medicine with anti-inflammatory and anti-oxidant properties, may have an inhibitory effect on the UV B-induced photo-damage by blocking the relevant cytokine secretion and expression of p53-p21, c-fos, PCNA, and RPA genes. Biflavonoids isolated from Selaginella tamariscina significantly inhibited UV irradiation induced activity of MMP-1 in primary fibroblasts [50 Kim, Chang, Moon, Lee, Oh, Woo, 2008]. Ziyuglycoside extracted from Sanguisorba officinalis root increased the expression of type I collagen in a dose-dependent manner and was found to decrease wrinkle formation in a clinical study. Triphlorethol-A, derived from Ecklonia cava exhibited antioxidative properties in human keratinocytes that involved the inhibition of MMP-1 via ERK and AP-1 inhibition. Topical application of bacterial sphingomyelinase from Streptococcus thermophiles increased skin ceramide levels in aged subjects and improved the skin lipid barrier and skin resistance against aging-associated xerosis. Resveratrol, a compound found in red wine can slow down aging and extend the lifespan of laboratory animals by activating members of the sirtuin gene family and transcription coactivators such as PGC-1a. It was also suggested that resveratrol can help to slow down skin aging. Hyaluronidase, an enzyme hydrolyzing glycoseaminoglycans, including hyaluronan, is important in conditions such as osteoarthritis and skin aging. Topical treatment with extracts from high phenolic Sorghum bran varieties was found to decrease hyaluronidase activity. Many of these treatments can be used in combination with esthetic dermatological procedures, such as laser resurfacing, other laser treatments, botulinum toxin injections, hyaluronic acid, calcium hydroxylapatite, and other dermal filler injections, chemical peels, and dermoabrasion.

Objective evaluation of the severity of aging phenotype is sometimes challenging, considering the many and varied social attitudes toward attractiveness and aging. Dermal aging is perhaps most noticeable on the human face followed by the hands. To minimize human subjectivity, computer software has been developed that estimates the age of individuals from digital facial images [61, 62]. Scoring systems were created that can quantify the age from the extent and severity of wrinkles and other signs. Biophysical models have been applied to model wrinkle development on the human face. A noninvasive method for evaluating skin aging based on near-infrared diffuse reflectance (NIR-DR) spectroscopy was also developed to study chronological and photoaging. These imaging, biophysical, computer modeling, and informatics methods can help to evaluate dermo-cosmetics' effectiveness in improving the skin phenotype.

In addition to skin biopsies, cultured fibroblasts, keratinocytes and sebocytes can be used as in vitro model systems for skin aging. Reconstructed skin models were also developed and used for the study of protein glycation. Animals and knockout mice can be used for identifying single genes that are key in skin aging (for example, NF-kappa B) and are also potential targets for cosmeceutical treatments.

Gene expression can be used for drug discovery and monitoring of treatment of aging and other dermal conditions. These other conditions include but are not limited to abrasion wound healing, psoriasis, atrophic dermatitis, alopecia and risk to skin cancer. Gene expression studies can help to separate disparate mechanisms of action of a treatment. For example, procollagen synthesis, a good marker for wound healing, can be separated from upregulation of proinflammatory cytokines, leukocyte adhesion molecules, MMPs, and other inflammatory markers. Gene expression studies can identify when members of a gene family are differentially regulated during aging. Gene expression profiling has been used successfully for the evaluation of genes that are up or downregulated in the aging human skin. To date, however, gene expression profiling has not been used to predict or monitor the effects of drugs, nutrients, vitamins, minerals, or naturally occurring compounds on reversing the skin aging process nor has it been used to identify networks of genes whose expression is essential in the skin aging process. This application describes a method using gene expression profiling and knowledge discovery tools to predict and monitor the effects of dermocosmetics, dietary supplements, and the constituents thereof on the delay, prevention, and reversal of human skin aging.

The results of four gene expression studies on human skin aging [72-75] were combined to determine which of the listed genes were up-regulated or down-regulated in these experiments. The gene names were corrected using the most recent gene names and symbol nomenclatures. Age-specific changes in gene expression were expressed as 2-based logarithm of the fold change. The mean values are presented in Table 1 and 2 for the up and down-regulated genes respectively.

The invention relates to compositions and methods used to reduce the visible signs of aging of the skin, particularly to the prevention and reduction of skin wrinkles and to the production and maintenance of youthful looking skin. The compositions and methods employ compounds that affect the expression of various genes within the skin tissue which genes are involved in chronological-induced and UV-induced skin damage. The present invention relates to compositions and methods that alter the expression of various genes that are involved in skin aging, for example genes involved in the biosynthesis or degradation of collagens and elastin, glycosaminoglycans and fibronectins or extracellular matrix (ECM) proteins such as collagen.

Synthesis and degradation of these compounds is directly related to the visible signs of aging, particularly the appearance of and the reduction in skin firmness and smoothness. As the amount of collagens, elastin, etc. are reduced within the skin, particularly in the dermis, the skin loses its firmness and smoothness, it becomes less hydrated, the outer layer of the skin becomes less supported by underlying layers thus sagging occurs and wrinkles appear.

This invention encompasses methods for reversing signs of skin aging and risk of skin cancer by resetting the expression of various genes, genetic networks, and cellular pathways in the human skin, primarily in the dermis, that change as a result of chronological or photo-induced aging process. Gene expression patterns, and the pathways they participate in, are restored to levels characteristic of a younger chronological age by treating the skin with specific combinations of natural compounds (for example, phytochemicals, nutrients, minerals, vitamins, etc.). Specific combinations of natural compounds are determined using informatic algorithms and high-throughput screening. Phytochemicals, nutrients, minerals, vitamins are well-known to those of skilled in the art.

The therapeutic compounds are applied topically, and/or in some embodiments are ingested. In certain embodiments, certain compounds are applied topically to the skin and other compounds are ingested, for example in the form of a pill, capsule, or powder dissolved in a liquid. The combination of topical and ingested compounds provides the desired benefit to the skin, altering gene expression and providing a genetic and biochemical environment that reduces the signs of aging (for example, wrinkles) or reduces the rate at which the signs of aging appear. Natural compounds are delivered to the dermis topically with dermocosmetics and internally with oral supplements. In some embodiments, combinations of natural compounds are claimed that produce synergistic effects on the expression of genes relevant to the reversal of skin aging and skin cancer risk reduction. Natural compounds can affect gene expression directly (for example, transcription factor agonists or antagonist) or indirectly (for example, noncoding RNAs, epigenetic modifications, signaling receptor agonists or antagonist). For example, lunasin, a chromatin remodeling soy peptide was found to modify epigenetic patterns. Natural compounds that produce synergistic effects in gene expression when administered both orally and topically are also claimed. For example, a chromatin remodeling peptide, lunasin applied topically may reach the dividing cells in the basal layer of the epidermis. While the same ingredient applied orally may reach many other tissues including the dermis of the skin. Also claimed are those genes, gene networks, noncoding RNAs and epigenetic modifications associated with chronologically younger or older skin listed in Tables 1-4.

### REFERENCES

The following patents, patent publications and other references are believed to be relevant to the invention:
1. Thompson-Torgerson C S, Holowatz L A, Kenney W L: Altered mechanisms of thermoregulatory vasoconstriction in aged human skin. Exerc Sport Sci Rev 2008, 36:122-127.
2. Holick M F: Vitamin D and sunlight: strategies for cancer prevention and other health benefits. Clin J Am Soc Nephrol 2008, 3:1548-1554.
3. Holick M F: Sunlight, UV-radiation, vitamin D and skin cancer: how much sunlight do we need? Adv Exp Med Biol 2008, 624:1-15.
4. Zouboulis C C, Chen W C, Thornton M J, Qin K, Rosenfield R: Sexual hormones in human skin. Horm Metab Res 2007, 39:85-95.
5. Sok J, Pineau N, Dalko-Csiba M, Breton L, Bernerd F: Improvement of the dermal epidermal junction in human reconstructed skin by a new c-xylopyranoside derivative. Eur J Dermatol 2008, 18:297-302.
6. Lee J Y, Kim Y K, Seo J Y, Choi C W, Hwang J S, Lee B G, Chang I S, Chung J H: Loss of elastic fibers causes skin wrinkles in sun-damaged human skin. J Dermatol Sci 2008, 50:99-107.
7. Koehler M J, Hahn S, Preller A, Elsner P, Ziemer M, Bauer A, Konig K, Buckle R, Fluhr J W, Kaatz M: Morphological skin aging criteria by multiphoton laser scanning tomography: non-invasive in vivo scoring of the dermal fibre network. Exp Dermatol 2008, 17:519-523.
8. Vleggaar D, Fitzgerald R: Dermatological implications of skeletal aging: a focus on supraperiosteal volumization for perioral rejuvenation. J Drugs Dermatol 2008, 7:209-220.
9. Tagami H: Functional characteristics of the stratum corneum in photoaged skin in comparison with those found in intrinsic aging. Arch Dermatol Res 2008, 300 Suppl 1:S1-6.
10. Giacomoni P U: Advancement in skin aging: the future cosmeceuticals. Clin Dermatol 2008, 26:364-366.
11. Serri R, Iorizzo M: Combating aging skin. Clin Dermatol 2008, 26:105.
12. Farage M A, Miller K W, Elsner P, Maibach H I: Functional and physiological characteristics of the aging skin. Aging Clin Exp Res 2008, 20:195-200.
13. Wollina U, Goldman A, Berger U, Abdel-Naser M B: Esthetic and cosmetic dermatology. Dermatol Ther 2008, 21:118-130.
14. Roberts W E: The Roberts Skin Type Classification System. J Drugs Dermatol 2008, 7:452-456.
15. Cosgrove M C, Franco O H, Granger S P, Murray P G, Mayes A E: Dietary nutrient intakes and skin-aging appearance among middle-aged American women. Am J Clin Nutr 2007, 86:1225-1231.
16. Moriwaki S, Takahashi Y: Photoaging and DNA repair. J Dermatol Sci 2008, 50:169-176.
17. Schroeder P, Lademann J, Darvin M E, Stege H, Marks C, Bruhnke S, Krutmann J: Infrared Radiation-Induced Matrix Metalloproteinase in Human Skin: Implications for Protection. J Invest Dermatol 2008.
18. Schroeder P, Haendeler J, Krutmann J: The role of near infrared radiation in photoaging of the skin. Exp Gerontol 2008, 43:629-632.
19. Morita A: Tobacco smoke causes premature skin aging. J Dermatol Sci 2007, 48:169-175.
20. Thornfeldt C R: Chronic inflammation is etiology of extrinsic aging. J Cosmet Dermatol 2008, 7:78-82.
21. Iddamalgoda A, Le Q T, Ito K, Tanaka K, Kojima H, Kido H: Mast cell tryptase and photoaging: possible involvement in the degradation of extra cellular matrix and basement membrane proteins. Arch Dermatol Res 2008, 300 Suppl 1:S69-76.
22. Sejersen H, Rattan S I: Dicarbonyl-induced accelerated aging in vitro in human skin fibroblasts. Biogerontology 2008.
23. Ortonne J P, Zartarian M, Verschoore M, Queille-Roussel C, Duteil L: Cellulite and skin aging: is there any interaction? J Eur Acad Dermatol Venereol 2008, 22:827-834.
24. Kaatz M, Elsner P, Koehler M J: Changes in skin topography during hormone therapy. Menopause 2008.
25. Helfrich Y R, Sachs D L, Voorhees J J: Overview of skin aging and photoaging. Dermatol Nurs 2008, 20:177-183; quiz 184.
26. Farage M A, Miller K W, Elsner P, Maibach H I: Intrinsic and extrinsic factors in skin aging: a review. Int J Cosmet Sci 2008, 30:87-95.
27. Uitto J: The role of elastin and collagen in cutaneous aging: intrinsic aging versus photoexposure. J Drugs Dermatol 2008, 7:s12-16.
28. Kwon O S, Yoo H G, Han J H, Lee S R, Chung J H, Eun H C: Photoaging-associated changes in epidermal proliferative cell fractions in vivo. Arch Dermatol Res 2008, 300:47-52.
29. Imokawa G: Recent advances in characterizing biological mechanisms underlying UV-induced wrinkles: a pivotal role of fibrobrast-derived elastase. Arch Dermatol Res 2008, 300 Suppl 1:S7-20.
30. Fisher G J, Varani J, Voorhees J J: Looking older: fibroblast collapse and therapeutic implications. Arch Dermatol 2008, 144:666-672.
31. Ogden S, Samuel M, Griffiths C E: A review of tazarotene in the treatment of photodamaged skin. Clin Intery Aging 2008, 3:71-76.
32. Kim H, Kim B, Um S, Lee J, Ryoo H, Jung H: Synthesis and in vitro biological activity of retinol retinoate, a novel hybrid retinoid derivative. Bioorg Med Chem 2008, 16:6387-6393.
33. Kalil C L, Fachinello F Z, Lamb F M, Comunello L N: Use of oral isotretinoin in photoaging therapy. Skinmed 2008, 7:10-14.
34. Jurzak M, Latocha M, Gojniczek K, Kapral M, Garncarczyk A, Pierzchala E: Influence of retinoids on skin fibroblasts metabolism in vitro. Acta Pol Pharm 2008, 65:85-91.
35. Bertin C, Zunino H, Lanctin M, Stamatas G N, Camel E, Robert C, Issachar N: Combined retinol-lactose-glycolic acid effects on photoaged skin: a double-blind placebo-controlled study. Int J Cosmet Sci 2008, 30:175-182.
36. Watson R E, Long S P, Bowden J J, Bastrilles J Y, Barton S P, Griffiths C E: Repair of photoaged dermal matrix by topical application of a cosmetic ‘antiaging’ product. Br J Dermatol 2008, 158:472-477.
37. Tresguerres J A, Kireev R, Tresguerres A F, Borras C, Vara E, Ariznavarreta C: Molecular mechanisms involved in the hormonal prevention of aging in the rat. J Steroid Biochem Mol Biol 2008, 108:318-326.
38. Hussain M, Phelps R, Goldberg D J: Clinical, histologic, and ultrastructural changes after use of human growth factor and cytokine skin cream for the treatment of skin rejuvenation. J Cosmet Laser Ther 2008, 10:104-109.
39. Nouveau S, Bastien P, Baldo F, de Lacharriere O: Effects of topical DHEA on aging skin: a pilot study. Maturitas 2008, 59:174-181.
40. Pineau N, Bernerd F, Cavezza A, Dalko-Csiba M, Breton L: A new C-xylopyranoside derivative induces skin expression of glycosaminoglycans and heparan sulphate proteoglycans. Eur J Dermatol 2008, 18:36-40.
41. Park H Y, Youm J K, Kwon M J, Park B D, Lee S H, Choi E H: K6PC-5, a novel sphingosine kinase activator, improves long-term ultraviolet light-exposed aged murine skin. Exp Dermatol 2008.
42. Youm J K, Jo H, Hong J H, Shin D M, Kwon M J, Jeong S K, Park B D, Choi E H, Lee S H: K6PC-5, a sphingosine kinase activator, induces anti-aging effects in intrinsically aged skin through intracellular Ca2+ signaling. J Dermatol Sci 2008, 51:89-102.
43. Lee J, Jung E, Yu H, Kim Y, Ha J, Kim Y S, Park D: Mechanisms of carvacrol-induced expression of type I collagen gene. J Dermatol Sci 2008.
44. Knott A, Koop U, Mielke H, Reuschlein K, Peters N, Muhr G M, Lenz H, Wensorra U, Jaspers S, Kolbe L, et al.: A novel treatment option for photoaged skin. J Cosmet Dermatol 2008, 7:15-22.
45. Tanaka H, Yamaba H, Kosugi N, Mizutani H, Nakata S: Fermentable metabolite of Zymomonas mobilis controls collagen reduction in photoaging skin by improving TGF-beta/Smad signaling suppression. Arch Dermatol Res 2008, 300 Suppl 1:S57-64.
46. Shim J S, Kwon Y Y, Hwang J K: The effects of panduratin A isolated from Kaempferia pandurata on the expression of matrix metalloproteinase-1 and type-1 procollagen in human skin fibroblasts. Planta Med 2008, 74:239-244.
47. Shim J S, Kwon Y Y, Han Y S, Hwang J K: Inhibitory Effect of Panduratin A on UV-induced Activation of Mitogen-Activated Protein Kinases (MAPKs) in Dermal Fibroblast Cells. Planta Med 2008.
48. Nsimba R Y, Kikuzaki H, Konishi Y: Ecdysteroids act as inhibitors of calf skin collagenase and oxidative stress. J Biochem Mol Toxicol 2008, 22:240-250.
49. Min W, Lin X F, Miao X, Wang B T, Yang Z L, Luo D: Inhibitory effects of baicalin on ultraviolet B-induced photo-damage in keratinocyte cell line. Am J Chin Med 2008, 36:745-760.
50. Lee C W, Choi H J, Kim H S, Kim D H, Chang I S, Moon H T, Lee S Y, Oh W K, Woo E R: Biflavonoids isolated from Selaginella tamariscina regulate the expression of matrix metalloproteinase in human skin fibroblasts. Bioorg Med Chem 2008, 16:732-738.
51. Kim Y H, Chung C B, Kim J G, Ko K I, Park S H, Kim J H, Eom S Y, Kim Y S, Hwang Y I, Kim K H: Anti-wrinkle activity of ziyuglycoside I isolated from a Sanguisorba officinalis root extract and its application as a cosmeceutical ingredient. Biosci Biotechnol Biochem 2008, 72:303-311.
52. Kang K A, Zhang R, Piao M J, Ko D O, Wang Z H, Lee K, Kim B J, Shin T, Park J W, Lee N H, et al.: Inhibitory effects of triphlorethol-A on MMP-1 induced by oxidative stress in human keratinocytes via ERK and AP-1 inhibition. J Toxicol Environ Health A 2008, 71:992-999.
53. Di Marzio L, Cinque B, Cupelli F, De Simone C, Cifone M G, Giuliani M: Increase of skin-ceramide levels in aged subjects following a short-term topical application of bacterial sphingomyelinase from Streptococcus thermophilus. Int J Immunopathol Pharmacol 2008, 21:137-143.
54. Baxter R A: Anti-aging properties of resveratrol: review and report of a potent new antioxidant skin care formulation. J Cosmet Dermatol 2008, 7:2-7.
55. Bralley E, Greenspan P, Hargrove J L, Hartle D K: Inhibition of hyaluronidase activity by select sorghum brans. J Med Food 2008, 11:307-312.
56. Berlin A L, Hussain M, Goldberg D J: Calcium hydroxylapatite filler for facial rejuvenation: a histologic and immunohistochemical analysis. Dermatol Surg 2008, 34 Suppl 1:S64-67.
57. Bruce S: Complementary effects of topical antiaging treatments in conjunction with aesthetic procedures. J Drugs Dermatol 2008, 7:s23-27.
58. Matts P J: New insights into skin appearance and measurement. J Investig Dermatol Symp Proc 2008, 13:6-9.
59. Fink B, Matts P J: The effects of skin colour distribution and topography cues on the perception of female facial age and health. J Eur Acad Dermatol Venereol 2008, 22:493-498.
60. Jakubietz R G, Kloss D F, Gruenert J G, Jakubietz M G: The aging hand. A study to evaluate the chronological aging process of the hand. J Plast Reconstr Aesthet Surg 2008, 61:681-686.
61. Tanaka H, Nakagami G, Sanada H, Sari Y, Kobayashi H, Kishi K, Konya C, Tadaka E: Quantitative evaluation of elderly skin based on digital image analysis. Skin Res Technol 2008, 14:192-200.
62. Staloff I A, Guan E, Katz S, Rafailovitch M, Sokolov A, Sokolov S: An in vivo study of the mechanical properties of facial skin and influence of aging using digital image speckle correlation. Skin Res Technol 2008, 14:127-134.
63. Shoshani D, Markovitz E, Monstrey S J, Narins D J: The modified Fitzpatrick Wrinkle Scale: a clinical validated measurement tool for nasolabial wrinkle severity assessment. Dermatol Surg 2008, 34 Suppl 1:S85-91; discussion S91.
64. Kuwazuru O, Saothong J, Yoshikawa N: Mechanical approach to aging and wrinkling of human facial skin based on the multistage buckling theory. Med Eng Phys 2008, 30:516-522.
65. Miyamae Y, Yamakawa Y, Kawabata M, Ozaki Y: A noninvasive method for assessing interior skin damage caused by chronological aging and photoaging based on near-infrared diffuse reflection spectroscopy. Appl Spectrosc 2008, 62:677-681.
66. Soroka Y, Maor Z, Leshem Y, Verochovsky L, Neuman R, Bregegere F M, Milner Y: Aged keratinocyte phenotyping: Morphology, biochemical markers and effects of Dead Sea minerals. Exp Gerontol 2008.
67. Makrantonaki E, Vogel K, Fimmel S, Oeff M, Seltmann H, Zouboulis C C: Interplay of IGF-I and 17beta-estradiol at age-specific levels in human sebocytes and fibroblasts in vitro. Exp Gerontol 2008.
68. Pageon H, Techer M P, Asselineau D: Reconstructed skin modified by glycation of the dermal equivalent as a model for skin aging and its potential use to evaluate anti-glycation molecules. Exp Gerontol 2008, 43:584-588.
69. Adler A S, Kawahara T L, Segal E, Chang H Y: Reversal of aging by NFkappaB blockade. Cell Cycle 2008, 7:556-559.
70. Warner R L, Bhagavathula N, Nerusu K, Hanosh A, McClintock S D, Naik M K, Johnson K J, Ginsburg I, Varani J: MDI 301, a nonirritating retinoid, improves abrasion wound healing in damaged/atrophic skin. Wound Repair Regen 2008, 16:117-124.
71. Oender K, Trost A, Lanschuetzer C, Laimer M, Emberger M, Breitenbach M, Richter K, Hintner H, Bauer J W: Cytokeratin-related loss of cellular integrity is not a major driving force of human intrinsic skin aging. Mech aging Dev 2008.
72. Lener T, Moll P R, Rinnerthaler M, Bauer J, Aberger F, Richter K: Expression profiling of aging in the human skin. Exp Gerontol 2006, 41:387-397.
73. Yoon I K, Kim H K, Kim Y K, Song I H, Kim W, Kim S, Baek S H, Kim J H, Kim J R: Exploration of replicative senescence-associated genes in human dermal fibroblasts by cDNA microarray technology. Exp Gerontol 2004, 39:1369-1378.
74. Funk W D, Wang C K, Shelton D N, Harley C B, Pagon G D, Hoeffler W K: Telomerase expression restores dermal integrity to in vitro-aged fibroblasts in a reconstituted skin model. Exp Cell Res 2000, 258:270-278.
75. Shelton D N, Chang E, Whittier P S, Choi D, Funk W D: Microarray analysis of replicative senescence. Curr Biol 1999, 9:939-945.

### Patents and Patent Publications

U.S. Pat. No. 6,569,624
US 2004/0142335
US 2006/0275294
US 2003/0152947
US 2005/0089914
US 2007/0059711
US 2005/0250137
US 2005/0250137
US 2005/0053637
U.S. Pat. No. 7,105,292
US 2007/0161022
U.S. Pat. No. 6,692,916
US 2006/0134663
US 2007/0148106
   All the above publications are incorporated by reference for all purposes.

TABLE 1
Table of age-related genes

| | | | | | |
|---|---|---|---|---|---|
| gene | Symbol | Ref1 (BJ) | Ref1 (C4) | Ref3 (MA) | ref2 (MA) |

A. Genes upregulated by skins aging

| | | | | | |
|---|---|---|---|---|---|
| matrix metaliopeptidase 3 (stromelysin 1, progelatinase) | MMP3 | 6.15 | 11.50 | (0.20) | |
| interleution 15 | IL15 | 3.50 | 6.10 | 3.30 | |
| chemoxine (C-C motif) ligand 2 | CCL2 | 5.95 | 3.50 | 2.95 | |
| plasminogen activator, tissue | PLAT | 4.00 | 3.15 | 5.30 | |
| WNT1 inducible signaling pathway protein 2 | WISP2 | | | | |
| toll-like receptor 4 | TLR4 | 4.85 | 4.40 | 1.75 | |
| Nitric oxide synthase 2A | NOS2A | | | | |
| cathepsin O | CTSO | 3.85 | 4.05 | 3.20 | |
| RAB3A, member RAS oncogene family | RAB3A | | | | |
| Small proline-rich protein 2B | SPRR2B | | | | |
| chemokine (C—X—C motif) ligand 1 | CXCL1 | 4.70 | 3.70 | 1.85 | 2.52 |
| (melanoma growth stimulating activity, alpha) | | | | | |
| chemokine (C—X—C motif) ligand 6 | CXCL6 | | | | 3.30 |
| (granulocyte chemetactic protein 2) | | | | | |
| superoxide dismutase 2, metochondrial (Mn) | SOD2 | 5.40 | 2.75 | 2.70 | |
| intercellular adhesion molecule 1 (CD54), | ICAM1 | | 2.45 | 4.55 | |
| human rhinevirus receptor | | | | | |
| serpin peptidase inhibitor, clade E (nexin plasminogen | SERPINE1 | | | | |
| activator inhibitor type 1), member 1 | | | | | |
| keratin 6A | KRT6A | | | | |
| insulin-like growth factor binding protein 5 | IGFBP5 | | | | 2.77 |
| Keratin 16 | KRT16 | | | | |
| stanniocalcin 1 | STC1 | | | | |
| matrix metaliopeptidase 10 (stromelysin 2) | MMP10 | 2.30 | 3.75 | 1.75 | |
| cyclin-dependent kinase inhibitor 1A (p21, Cip1) | CDKN1A | | | | |
| ubiquitin specific peptidase 53 | USP53 | | | | |
| S100 calcium binding protein A9 | S100A9 | | | | |
| NSCRA image clone2825070 | | | | | |
| growth arrest-specific 1 | GAS1 | | | | |
| insulin-like growth factor binding protein 2 | IGFBP2 | | | | |
| peptidylglycine alpha-amidating monooxygenase | PAM | | | | |
| Interleukin 1 beta | IL1B | 3.65 | | 1.25 | |
| DNA-damage-inducible transcript 3 | DDIT3 | | | | |
| Rho family GTPase 3 | RND3 | | | | |
| plasminogen activator, urokinase | PLAU | 3.05 | 3.25 | | |
| Fucosyltransferase 3 | FUT3 | | | | |
| peptidase inhibitor 3, skin-derived (SKALP) | PI3 | | | | |
| myxovirus (influenza virus) resistance 1, | MX1 | | | | 2.11 |
| interferon-inducible protein p78 (mouse) | | | | | |
| Cathepsin D | CTSD | | | | |
| similar to gb: MB1181 SODIUM/POTASSIUM- | | | | | 1.79 |
| TRANSPORTING ATPASE BETA-2 | | | | | |
| tumor necrosis factor, alpha-induced protein 6 | TNFAIP6 | | | | 1.70 |
| interleukin 6 (interferon, beta 2) | IL6 | | | | 1.43 |
| prcenkephalin | PENK | | | | 1.71 |
| Tumor protein D52-like 1 | TPD52L1 | | | | 1.46 |
| chemokine (C-C motif) ligand 8 | CCL8 | | | | 1.60 |
| Forkhead box O1A (rhabdcmyosarcoma) | FOXO1A | | | | |
| CD36 molecule (thrombospondin receptor) | CD36 | | | | 1.85 |
| Forkhead box C1 | FOXC1 | | | | |
| 2′,5′-oligoadenylate synthetase 1, 40/46 kDa | OAS1 | | | | 1.01 |
| S100 calcium binding protein A2 | S100A2 | | | | |
| chemokine (C-C motif) ligand 7 | CCL7 | | | | 1.52 |
| chemokine (C-C motif) ligand 11 | CCL11 | | | | 1.49 |
| matrix metaliopeptidase 1 (interstitial collagenase) | MMP1 | 1.85 | | 5.30 | (1.15) |
| Protease, serine 3 | PRSS3 | | | | |
| solute carrier family 39 (zinc transporter), member 8 | SLC39A8 | | | | 1.52 |
| Interferon, alpha-inducible protein 6 | IFI6 | | | | 2.13 |
| Rho GDP dissociation inhibitor (GDI) beta | ARHGDIB | | | | |
| Antigen identified by monoclonal antibody Ki-67 | MKi67 | | | | |
| cDNA FLJ11558 | | | | | 1.34 |
| Myosin, light polypeptide kinase | MYLK | | | | |
| Stennin | SNN | | | | |
| Kallikrein 13 | KLK13 | | | | |
| dipeptidyl-peptidase 4 | DPP4 | | | | 1.25 |
| (CD26, adenosine deaminase complexing protein 2) | | | | | |
| keratin 19 | KRT19 | | | | 2.13 |
| Comeodesmosin | CDSN | | | | |
| GC/Giswitch 2 | GCG2 | | | | 1.55 |
| keratin 14 | KRT14 | | | | 1.25 |
| mitochondrially encoded cytochrome c oxidase II | MT-CO2 | | | | 1.41 |
| Crystaline, alpha B | CRYAB | | | | |
| Rho GTPase-activating protein | GRIT | | | | |
| major histocompatibility complex, class I, C | HLA-C | | | | 1.23 |
| natriuretic peptide receptor A/guanylate cyclase A | NPR1 | | | | 1.23 |
| (atrionatriuretic peptide receptor A) | | | | | |
| Collagen, type 27, alpha 1 | COL27A1 | | | | |
| major histocompatibility complex, class I, B | HLA-B | | | | 1.17 |
| MAP/microtubule affinity-regulating kinase 2 | MARK2 | | | | |
| Neurobeachin-like 2 | NBEAL2 | | | | |
| fibronectin 1 | FN1 | | | | 1.15 |
| interleukin 17C | IL17C | | | | |
| Hook homolog 1 (Drosophila) | HOOK1 | | | | |
| beta-2-microglobulin | B2M | | | | 1.12 |
| interferon-induced protein with tetratricopeptide repeats 1 | IFIT1 | | | | 1.60 |
| cadherin 2, type 1, N-cadherin (neuronal) | CDH2 | | | | 1.10 |
| complement factor B | CFB | | | | 1.40 |
| BCL2/adenovirus E18 19 kDa interacting protein 3 | BNIP3 | | | | |
| Claudin 8 | CLDN8 | | | | |
| Ets homologcus factor | EHF | | | | |
| cDNA clone CS0DE014YK0 | | | | | |
| Signal transducer and activator of transcription 3 | STAT3 | | | | |
| interferon-induced protein 44-like | IFI44L | | | | 1.05 |
| S100 calcium binding protein A7 | S100A7 | | | | |
| Squalene epoxidase | SQLE | | | | |
| phospholipase A2 receptor 1, 180 kDa | PLA2R1 | | | | 1.24 |
| adrenergic, alpha-1D-, receptor | ADRA1D | | | | 1.03 |
| major histocompatibility complex, class I, A | HLA-A | | | | 1.02 |
| Cathepsin L | CTSL1 | | | | |
| Secreted frizzled related protein 4 | SFRP4 | | | | |
| Coronia, actin binding protein, 2A | CORO2A | | | | |
| UDP-gal, polypeptide 6 | S4GALT6 | | | | |
| Transforming growth factor, alpha | TGFA | | | | |
| Basonucin 1 | BNC1 | | | | |
| Low density lipoprotein receptor | LDLR | | | | |
| Hexokinase 2 | HK | | | | 1.75 |
| Cbp/p300-interacting transactivator 1 | CITED1 | | | | |
| cDNA clone FLJ33158 | | | | | |
| Farnesyl-diphosphate farnesyltransferase 1 | FDFT1 | | | | |
| proteasome subunit, beta type, 9 | PSMB9 | | | | |
| LOC147645 | LOC147645 | | | | |
| UDP-glucose ceramide glucosyltransferase | UGCG | | | | |
| ISG15 ubiquitin-like modifier | ISG15 | | | | 1.22 |
| insulin induced gene 1 | INSIG1 | | | | |
| Kynureninase (L-kynurenine hydrotase) | KYNU | | | | |
| Actin binding LIM protein 1 | ABLIM1 | | | | |
| Serine proteinase inhibitor, clade B, member 2 | SERPINB2 | | | | |
| MAX dimerization protein 1 | MKD1 | | | | |
| brain-specific angiogenesis inhibitor 3 | BAI3 | | | | 1.73 |
| homeobox containing 1 | HMBOX1 | | | | |
| RAS p21 protein activator 4 | RASA4 | | | | |
| Myosin 1E | MYO1E | | | | |
| Apolipoprotein D | APOD | | | | |
| Suppression of tumorigenicity 14 | ST14 | | | | |
| Vestigial like 4 (Drosophila) | VGLL4 | | | | |
| Cyclin E1 | CCNE1 | | | | |
| ADAM metaliopeptidase with thrombospondin type 1 motif, 1 | ADAMTS1 | | | | 1.20 |
| Metal-regulatory transcription factor 1 | MTF1 | | | | |
| dickkopt homolog 1 (Xenopus laevis) | DKK1 | | | | 1.10 |
| erythrocyte membrane protein band 4.1-like 3 | EPB41L3 | | | | 1.48 |

B. Genes down-regulated by skin aging

| elastin (supravateular aortic stenosts, | ELN | (6.95) | (4.50) | (10.65) | (1.09) |
|---|---|---|---|---|---|
| Williams-Beuren syndrome) | | | | | |
| Delta-like 1 homolog (Drosophila) | DLK1 | | | | |
| keratin 7 | NRT7 | | | | |
| collagen, type I, alpha 1 | COL1A1 | | | | |
| integrin, alpha 1 | ITGA1 | | | | |
| cnemokine (C—X—C motif) receptor 7 | CXCR7 | | | | (2.46) |
| integrin, alpha 4 (antigen CD49D, alpha 4 subunit of VLA-4 receptor) | ITGA4 | | | | (1.89) |
| collagen, type III, alpha 1 | COL3A1 | | | | |
| (Enters-Dantos syndrome type IV, autosomat dominant) | | | | | |
| runs-related transcription factor 2 | RUNX2 | | | | (1.89) |
| intereukin 22 receptor, alpha 2 | IL22RA2 | | | | |
| BRCA1 associated protein-1 (ubiquitin carboxy-terminal hydrolase) | SAP1 | | | | (3.47) |
| FB3 osteosarcoma viral oncogene homolog 8 | FOS8 | | | | |
| Nuciear receptor subfamily 4, group A, member 1 | NR4A1 | | | | |
| v-Fos FB3 osteosarcoma viral oncogene homolog | FOS | | | | |
| calpain 6 | CAPN6 | | | | (1.60) |
| Keratin 2A | KRT2 | | | | |
| NADH dehydrogenase (ubiquincae) flavoprotein 2, 24 kDa | NDUFV2 | | | | (1.56) |
| zinc finger, MYMD domain containing 11 isoform a | ZMYND11 | | | | (1.56) |
| centromere protein F, 350/400 ka (mitosin) | CENPF | | | | (1.40) |
| insutin-like growth factor 2 (somatomedin A) | IGF2 | | | | (1.64) |
| KIAA0564 | KIAA0564 | | | | (1.45) |
| Glypican 6 | GPC6 | | | | |
| CUG triplet repeat, RNA binding protein 2 | CUGBP2 | | | | (1.69) |
| Kinesin family member 4A | KSF4A | | | | (1.46) |
| Syntaion 6 | STX6 | | | | |
| dermato [symbol] tia | DPT | | | | (1.32) |
| myosin, heavy chain 11, smooth muscle | MYH11 | | | | (1.22) |
| SWI/SNF subfamily a, member 1 | SMARCA1 | | | | |
| Glypican 3 | GPC3 | | | | |
| aggrecan | ACAN | | | | (1.15) |
| Chemokine (C-C motif) ligand 21 | CCL21 | | | | |
| fibrillin 2 (congenital contrectural arachnodectyly) | FBN2 | | | | (1.47) |
| immunogiobuin superfamily containing leucine-rich repeat | ISLR | | | | (1.51) |
| RAB3 GTPase activating protein subunit 2 (non-catalytic) | RAB3GAP2 | | | | (1.79) |
| transmembrane protein 132C | TMEM132C | | | | |
| choroideremia (Rab escort protein 1) | CHM | | | | (2.12) |
| stearoyl-CoA desaturase (delta-9-desaturase) | SCD | | | | (1.64) |
| Discs, large (Drosophila) homolog 1 | DLG1 | | | | (2.06) |
| neurotrimin | HNT | | | | (1.19) |
| Meningioma (disrupted in balanced transtocation) | MN1 | | | | (1.09) |
| Rap guanine nuclectioe exchange factor (GEF) 5 | RAPGEF5 | | | | |
| Fatty acid binding protein 7, brain | FABP7 | | | | |
| Tumor protein p73-like | TP73L | | | | |
| Fibronectin leucine rich transmembrane protein 2 | FLRT2 | | | | |
| PDZX1 interacting protein 1 | PDZK1IP1 | | | | |
| wingtess-type MMTV integration site family member 2 | WNT2 | | | | (1.40) |
| cyclin A2 | CCNA2 | | | | (1.00) |
| MYC associated factor X | MAX | | | | |
| Latrophilin 3 | LPHN3 | | | | |
| Thrombospondin 1 | THBS1 | | | | |
| Autism susceptibility candidate 2 | AUTS2 | | | | |
| Hexckinase 1 | HK1 | | | | |
| NK2 transcription factor related (Drosophila) | NKX2-2 | | | | |
| Transglutaminase 3 | TGM3 | | | | |
| c-Myc | MYC | | | | |
| Caspase 1, apoptosis-reiated cysteine protease | CASP1 | | | | |
| Transmembrane 6 superfamily member 1 | TM6SF1 | | | | |
| v-ion sarcoma virus 17 oncogene homolog (avian) | JUN | | | | |
| protocadherin gamma | PCDHG | | | | (1.64) |
| Slit homolog 2 (Drosophila) | SUT2 | | | | |
| ion 5 proto-oncogene | JUNB | | | | |
| citrate synthase | CS | | | | (1.64) |
| interferon regulatory factor 1 | IRF1 | | | | |
| CD8 antigen, alpha polypeptide (p32) | CDSA | | | | |
| Kinesin famliy member 9 | KJF9 | | | | |
| myeloid/lymphoid or mixed-lineage leukemia | MLLT4 | | | | |
| (trithorax homolog, Drosophila); transtocated to, 4 | | | | | |
| cysteine-rich protein 1 (intestinal) | CRIP1 | | | | (1.43) |
| zinc finger, NFX1-type containing 1 | ZNFX1 | | | | |
| SRY (sex determining region Y)-box 9 | SOX9 | | | | (1.43) |
| (campometic dysplasia, autosomal sex-reversat) | | | | | |
| Osteoclast simulating factor 1 | OSTF1 | | | | |
| far upstream element (FUSE) binding protein 2 | FUBP1 | | | | (1.25) |
| endegin (Oster-Rendu-Weber syndrome 1) | ENG | | | | (1.56) |
| Phospholipase D1 | PLD1 | | | | |
| protein kinase N2 | PKN2 | | | | (1.12) |
| malignant fibrous histiocytoma amplified sequence 1 | MFHAS1 | | | | (1.15) |
| cyclin 1 | CCN1 | | | | (1.47) |
| GLE1 RNA export mediator homolog (yeast) | GLE1 | | | | (1.18) |
| Chemokine (C-C motif) ligand 18 | CCL12 | | | | |
| early endosome antigen 1 | EEA1 | | | | (1.47) |
| PWP2 periodic tryptophan protein homolog (yeast) | PWP2 | | | | (1.12) |
| growth arrest-specific 6 | GAS6 | | | | (1.12) |
| protein reguiator of cytokinesis 1 | PRC1 | | | | (1.03) |
| aldehyde dehydrogenase 2 family (mitochondrial) | ALDH2 | | | | (1.06) |
| Cyclin D1 | CCND1 | | | | 1.10 |
| insulin-like growth factor binding protein 3 | IGFBP3 | | | | 1.96 |
| gene | ref2 (PCR) | ref3 (MA) | ref4 (MA) | ref4 (PCR) | average |

A. Genes upregulated by skins aging

| | | | | | |
|---|---|---|---|---|---|
| matrix metaliopeptidase 3 (stromelysin 1, progelatinase) | | 2.17 | | | 4.90 |
| interleution 15 | | | | | 4.30 |
| chemoxine (C-C motif) ligand 2 | | 3.25 | | | 3.91 |
| plasminogen activator, tissue | | 2.70 | | | 3.79 |
| WNT1 inducible signaling pathway protein 2 | | | 2.16 | 5.14 | 3.65 |
| toll-like receptor 4 | | 2.91 | | | 3.48 |
| Nitric oxide synthase 2A | | | 1.63 | 5.32 | 3.47 |
| cathepsin O | | 2.58 | | | 3.42 |
| RAB3A, member RAS oncogene family | | 3.29 | | | 3.29 |
| Small proline-rich protein 2B | | | 2.67 | 3.57 | 3.12 |
| chemokine (C—X—C motif) ligand 1 | 2.80 | 3.00 | | | 3.09 |
| (melanoma growth stimulating activity, alpha) | | | | | |
| chemokine (C—X—C motif) ligand 6 | 2.86 | | | | 3.08 |
| (granulocyte chemetactic protein 2) | | | | | |
| superoxide dismutase 2, metochondrial (Mn) | | 3.05 | 1.45 | | 3.06 |
| intercellular adhesion molecule 1 (CD54), | | 2.00 | | | 3.00 |
| human rhinevirus receptor | | | | | |
| serpin peptidase inhibitor, clade E (nexin plasminogen | | 2.81 | | | 2.81 |
| activator inhibitor type 1), member 1 | | | | | |
| keratin 6A | | | 1.08 | 4.44 | 2.76 |
| insulin-like growth factor binding protein 5 | 2.66 | 2.56 | | | 2.66 |
| Keratin 16 | | | 1.60 | 3.58 | 2.59 |
| stanniocalcin 1 | | 2.58 | | | 2.58 |
| matrix metaliopeptidase 10 (stromelysin 2) | | 2.32 | | | 2.53 |
| cyclin-dependent kinase inhibitor 1A (p21, Cip1) | | 2.46 | | | 2.46 |
| ubiquitin specific peptidase 53 | | 2.46 | | | 2.46 |
| S100 calcium binding protein A9 | | | 1.97 | 2.89 | 2.43 |
| NSCRA image clone2825070 | | | 2.34 | | 2.34 |
| growth arrest-specific 1 | | 2.32 | | | 2.32 |
| insulin-like growth factor binding protein 2 | | 2.32 | | | 2.32 |
| peptidylglycine alpha-amidating monooxygenase | | 2.32 | | | 2.32 |
| Interleukin 1 beta | | 1.81 | | | 2.24 |
| DNA-damage-inducible transcript 3 | | 2.00 | | | 2.00 |
| Rho family GTPase 3 | | 2.00 | | | 2.00 |
| plasminogen activator, urokinase | | 1.58 | | | 1.97 |
| Fucosyltransferase 3 | | | 1.27 | 2.59 | 1.93 |
| peptidase inhibitor 3, skin-derived (SKALP) | | | 1.17 | 2.63 | 1.90 |
| myxovirus (influenza virus) resistance 1, | 1.56 | | | | 1.83 |
| interferon-inducible protein p78 (mouse) | | | | | |
| Cathepsin D | | | 0.78 | 2.84 | 1.81 |
| similar to gb: MB1181 SODIUM/POTASSIUM- | | | | | 1.79 |
| TRANSPORTING ATPASE BETA-2 | | | | | |
| tumor necrosis factor, alpha-induced protein 6 | | | | | 1.70 |
| interleukin 6 (interferon, beta 2) | 1.24 | 2.32 | | | 1.66 |
| prcenkephalin | 1.57 | | | | 1.64 |
| Tumor protein D52-like 1 | 1.77 | | | | 1.62 |
| chemokine (C-C motif) ligand 8 | | | | | 1.60 |
| Forkhead box O1A (rhabdcmyosarcoma) | | | 1.59 | | 1.59 |
| CD36 molecule (thrombospondin receptor) | 1.34 | | | | 1.59 |
| Forkhead box C1 | | | 1.57 | | 1.57 |
| 2′,5′-oligoadenylate synthetase 1, 40/46 kDa | 2.08 | | | | 1.54 |
| S100 calcium binding protein A2 | | | 1.23 | 1.82 | 1.53 |
| chemokine (C-C motif) ligand 7 | | | | | 1.52 |
| chemokine (C-C motif) ligand 11 | | | | | 1.49 |
| matrix metaliopeptidase 1 (interstitial collagenase) | (0.07) | | | | 1.48 |
| Protease, serine 3 | | | 1.18 | 1.72 | 1.45 |
| solute carrier family 39 (zinc transporter), member 8 | 1.32 | | | | 1.42 |
| Interferon, alpha-inducible protein 6 | 0.58 | | | | 1.36 |
| Rho GDP dissociation inhibitor (GDI) beta | | | 1.35 | | 1.35 |
| Antigen identified by monoclonal antibody Ki-67 | | | 1.34 | | 1.34 |
| cDNA FLJ11558 | | | | | 1.34 |
| Myosin, light polypeptide kinase | | | 1.33 | | 1.33 |
| Stennin | | | 1.47 | 1.14 | 1.31 |
| Kallikrein 13 | | | 1.29 | | 1.29 |
| dipeptidyl-peptidase 4 | 1.32 | | | | 1.29 |
| (CD26, adenosine deaminase complexing protein 2) | | | | | |
| keratin 19 | 0.42 | | | | 1.27 |
| Comeodesmosin | | | 1.26 | | 1.26 |
| GC/Giswitch 2 | 1.38 | | 1.34 | 0.74 | 1.25 |
| keratin 14 | | | | | 1.25 |
| mitochondrially encoded cytochrome c oxidase II | 1.07 | | | | 1.24 |
| Crystaline, alpha B | | | 0.99 | 1.49 | 1.24 |
| Rho GTPase-activating protein | | | 1.23 | | 1.23 |
| major histocompatibility complex, class I, C | | | | | 1.23 |
| natriuretic peptide receptor A/guanylate cyclase A | | | | | 1.23 |
| (atrionatriuretic peptide receptor A) | | | | | |
| Collagen, type 27, alpha 1 | | | 1.19 | | 1.19 |
| major histocompatibility complex, class I, B | | | | | 1.17 |
| MAP/microtubule affinity-regulating kinase 2 | | | 1.16 | | 1.16 |
| Neurobeachin-like 2 | | | 1.16 | | 1.16 |
| fibronectin 1 | | | | | 1.15 |
| interleukin 17C | | | 1.15 | | 1.15 |
| Hook homolog 1 (Drosophila) | | | 1.12 | | 1.12 |
| beta-2-microglobulin | | | | | 1.12 |
| interferon-induced protein with tetratricopeptide repeats 1 | 0.61 | | | | 1.11 |
| cadherin 2, type 1, N-cadherin (neuronal) | | | | | 1.10 |
| complement factor B | 0.81 | | | | 1.10 |
| BCL2/adenovirus E18 19 kDa interacting protein 3 | | | 1.08 | | 1.08 |
| Claudin 8 | | | 1.08 | | 1.08 |
| Ets homologcus factor | | | 1.07 | | 1.07 |
| cDNA clone CS0DE014YK0 | | | 1.07 | | 1.07 |
| Signal transducer and activator of transcription 3 | | | 1.08 | 1.05 | 1.07 |
| interferon-induced protein 44-like | | | | | 1.06 |
| S100 calcium binding protein A7 | | | 1.06 | | 1.06 |
| Squalene epoxidase | | | 1.05 | | 1.05 |
| phospholipase A2 receptor 1, 180 kDa | 0.86 | | | | 1.05 |
| adrenergic, alpha-1D-, receptor | | | | | 1.03 |
| major histocompatibility complex, class I, A | | | | | 1.02 |
| Cathepsin L | | | 1.01 | | 1.01 |
| Secreted frizzled related protein 4 | | | 1.01 | | 1.01 |
| Coronia, actin binding protein, 2A | | | 0.99 | | 0.99 |
| UDP-gal, polypeptide 6 | | | 0.99 | | 0.99 |
| Transforming growth factor, alpha | | | 0.97 | | 0.97 |
| Basonucin 1 | | | 0.97 | | 0.97 |
| Low density lipoprotein receptor | | | 0.96 | | 0.96 |
| Hexokinase 2 | 0.66 | | | | 0.96 |
| Cbp/p300-interacting transactivator 1 | | | 0.81 | 1.10 | 0.95 |
| cDNA clone FLJ33158 | | | 0.95 | | 0.95 |
| Farnesyl-diphosphate farnesyltransferase 1 | | | 0.91 | | 0.91 |
| proteasome subunit, beta type, 9 | | | 0.91 | | 0.91 |
| LOC147645 | | | 0.89 | | 0.89 |
| UDP-glucose ceramide glucosyltransferase | | | 0.87 | 0.90 | 0.89 |
| ISG15 ubiquitin-like modifier | 0.54 | | | | 0.88 |
| insulin induced gene 1 | | | 0.86 | | 0.86 |
| Kynureninase (L-kynurenine hydrotase) | | | 0.86 | | 0.86 |
| Actin binding LIM protein 1 | | | 1.16 | 0.56 | 0.86 |
| Serine proteinase inhibitor, clade B, member 2 | | | 0.85 | | 0.85 |
| MAX dimerization protein 1 | | | 0.85 | | 0.85 |
| brain-specific angiogenesis inhibitor 3 | (0.07) | | | | 0.83 |
| homeobox containing 1 | | | 0.82 | | 0.82 |
| RAS p21 protein activator 4 | | | 0.81 | | 0.81 |
| Myosin 1E | | | 0.80 | | 0.80 |
| Apolipoprotein D | | | 0.79 | | 0.79 |
| Suppression of tumorigenicity 14 | | | 0.77 | | 0.77 |
| Vestigial like 4 (Drosophila) | | | 0.79 | 0.75 | 0.77 |
| Cyclin E1 | | | 0.77 | | 0.77 |
| ADAM metaliopeptidase with thrombospondin type 1 motif, 1 | 0.06 | | | | 0.63 |
| Metal-regulatory transcription factor 1 | | | 0.62 | | 0.62 |
| dickkopt homolog 1 (Xenopus laevis) | 0.07 | | | | 0.58 |
| erythrocyte membrane protein band 4.1-like 3 | (1.06) | | | | 0.21 |

B. Genes down-regulated by skin aging

| | | | | | |
|---|---|---|---|---|---|
| elastin (supravateular aortic stenosts, | | (2.58) | | | (5.14) |
| Williams-Beuren syndrome) | | | | | |
| Delta-like 1 homolog (Drosophila) | | | (1.28) | (6.98) | (4.13) |
| keratin 7 | | (2.26) | | | (2.26) |
| collagen, type I, alpha 1 | | (2.17) | | | (2.17) |
| integrin, alpha 1 | | (2.00) | | | (2.00) |
| cnemokine (C—X—C motif) receptor 7 | (1.51) | | | | (1.96) |
| integrin, alpha 4 (antigen CD49D, alpha 4 subunit of VLA-4 receptor) | | | | | (1.89) |
| collagen, type III, alpha 1 | | (1.89) | | | (1.89) |
| (Enters-Dantos syndrome type IV, autosomat dominant) | | | | | |
| runs-related transcription factor 2 | (1.64) | | | | (1.77) |
| intereukin 22 receptor, alpha 2 | | | (1.26) | (2.27) | (1.76) |
| BRCA1 associated protein-1 (ubiquitin carboxy-terminal hydrolase) | (0.04) | | | | (1.76) |
| FB3 osteosarcoma viral oncogene homolog 8 | | | (1.71) | | (1.71) |
| Nuciear receptor subfamily 4, group A, member 1 | | | (1.06) | (2.37) | (1.71) |
| v-Fos FB3 osteosarcoma viral oncogene homolog | | | (1.31) | (2.08) | (1.69) |
| calpain 6 | | | | | (1.60) |
| Keratin 2A | | | (1.20) | (1.92) | (1.56) |
| NADH dehydrogenase (ubiquincae) flavoprotein 2, 24 kDa | | | | | (1.56) |
| zinc finger, MYMD domain containing 11 isoform a | | | | | (1.56) |
| centromere protein F, 350/400 ka (mitosin) | (1.51) | | | | (1.46) |
| insutin-like growth factor 2 (somatomedin A) | (1.84) | | (1.03) | (1.28) | (1.45) |
| KIAA0564 | | | | | (1.45) |
| Glypican 6 | | | (0.93) | (1.77) | (1.35) |
| CUG triplet repeat, RNA binding protein 2 | (1.00) | | | | (1.34) |
| Kinesin family member 4A | (1.18) | | | | (1.25) |
| Syntaion 6 | | | (1.26) | | (1.26) |
| dermato [symbol] tia | (1.12) | | | | (1.22) |
| myosin, heavy chain 11, smooth muscle | | | | | (1.22) |
| SWI/SNF subfamily a, member 1 | | | (1.19) | | (1.19) |
| Glypican 3 | | | (1.16) | | (1.16) |
| aggrecan | | | | | (1.15) |
| Chemokine (C-C motif) ligand 21 | | | (0.95) | (1.33) | (1.14) |
| fibrillin 2 (congenital contrectural arachnodectyly) | (0.74) | | | | (1.11) |
| immunogiobuin superfamily containing leucine-rich repeat | (0.69) | | | | (1.10) |
| RAB3 GTPase activating protein subunit 2 (non-catalytic) | (0.42) | | | | (1.10) |
| transmembrane protein 132C | | | (1.10) | | (1.10) |
| choroideremia (Rab escort protein 1) | (0.07) | | | | (1.10) |
| stearoyl-CoA desaturase (delta-9-desaturase) | (0.51) | | | | (1.08) |
| Discs, large (Drosophila) homolog 1 | (0.07) | | | | (1.07) |
| neurotrimin | (0.84) | | | | (1.06) |
| Meningioma (disrupted in balanced transtocation) | (1.00) | | | | (1.04) |
| Rap guanine nuclectioe exchange factor (GEF) 5 | | | (1.04) | | (1.04) |
| Fatty acid binding protein 7, brain | | | (1.03) | | (1.03) |
| Tumor protein p73-like | | | (1.03) | | (1.03) |
| Fibronectin leucine rich transmembrane protein 2 | | | (1.01) | | (1.01) |
| PDZX1 interacting protein 1 | | | (1.01) | | (1.01) |
| wingtess-type MMTV integration site family member 2 | (0.62) | | | | (1.01) |
| cyclin A2 | | | | | (1.00) |
| MYC associated factor X | | | (1.00) | | (1.00) |
| Latrophilin 3 | | | (1.21) | (0.75) | (0.98) |
| Thrombospondin 1 | | | (0.96) | | (0.96) |
| Autism susceptibility candidate 2 | | | (1.02) | (0.88) | (0.95) |
| Hexckinase 1 | | | (0.95) | | (0.95) |
| NK2 transcription factor related (Drosophila) | | | (0.94) | | (0.94) |
| Transglutaminase 3 | | | (0.94) | | (0.94) |
| c-Myc | | | (0.82) | (1.05) | (0.94) |
| Caspase 1, apoptosis-reiated cysteine protease | | | (0.93) | | (0.93) |
| Transmembrane 6 superfamily member 1 | | | (0.90) | | (0.90) |
| v-ion sarcoma virus 17 oncogene homolog (avian) | | | (0.85) | | (0.85) |
| protocadherin gamma | (0.10) | | | | (0.87) |
| Slit homolog 2 (Drosophila) | | | (0.91) | 0.81) | (0.86) |
| ion 5 proto-oncogene | | | (0.77) | 0.94) | (0.85) |
| citrate synthase | (0.01) | | | | (0.83) |
| interferon regulatory factor 1 | | | (0.91) | (0.73) | (0.82) |
| CD8 antigen, alpha polypeptide (p32) | | | (0.82) | (0.82) | (0.82) |
| Kinesin famliy member 9 | | | (0.82) | | (0.82) |
| myeloid/lymphoid or mixed-lineage leukemia | | | (0.81) | | (0.81) |
| (trithorax homolog, Drosophila); transtocated to, 4 | | | | | |
| cysteine-rich protein 1 (intestinal) | (0.15) | | | | (0.79) |
| zinc finger, NFX1-type containing 1 | | | (0.79) | | (0.79) |
| SRY (sex determining region Y)-box 9 | (0.14) | | | | (0.79) |
| (campometic dysplasia, autosomal sex-reversat) | | | | | |
| Osteoclast simulating factor 1 | | | (0.77) | | (0.77) |
| far upstream element (FUSE) binding protein 2 | (0.25) | | | | (0.75) |
| endegin (Oster-Rendu-Weber syndrome 1) | 0.06 | | | | (0.75) |
| Phospholipase D1 | | | (0.85) | (0.64) | (0.74) |
| protein kinase N2 | (0.32) | | | | (0.72) |
| malignant fibrous histiocytoma amplified sequence 1 | (0.29) | | | | (0.72) |
| cyclin 1 | 0.04 | | | | (0.72) |
| GLE1 RNA export mediator homolog (yeast) | (0.25) | | | | (0.71) |
| Chemokine (C-C motif) ligand 18 | | | (0.70) | | (0.70) |
| early endosome antigen 1 | 0.16 | | | | (0.66) |
| PWP2 periodic tryptophan protein homolog (yeast) | (0.07) | | | | (0.66) |
| growth arrest-specific 6 | — | | | | (0.56) |
| protein reguiator of cytokinesis 1 | (0.06) | | | | (0.54) |
| aldehyde dehydrogenase 2 family (mitochondrial) | 0.10 | | | | (0.48) |
| Cyclin D1 | | | (1.20) | (0.81) | (0.30) |
| insulin-like growth factor binding protein 3 | | | (2.09) | (2.28) | (0.14) |

Notes:
All gene expression changes are expressed in 2-based logarithms of the change. Therefore a +1 value stands for 2-fold over-expression and −1 stands for 2-fold suppression. Negative numbers are listed in parentheses. [symbol]
indicates data missing or illegible when filed

### REFERENCES

1 In Kyung Yoon et al. Microarray analysis of replicative senescence 1999 Current Biology, Vol 9 No 17
2 In Kyung Yoon et al. Exploration of replicative senescence-associated genes in human dermal 2004 Experimental Gerontology 39, 1369-1378
3 Walter D. Funk et al. Telomerase Expression Restores Dermal Integrity to in Vitro-Aged Fibrob 2000 Experimental Cell Research 258, 270-278
4 Thomas Lener et al. Expression profiling of aging in the human skin 2006 Experimental Gerontology 41, 387-397

TABLE 2
Table of aging-related microRNAs

| | |
|---|---|
| hsa-miR-181d | 29 |
| hsa-miR-200b | 26 |
| hsa-miR-548c-5p | 26 |
| hsa-miR-181b | 25 |
| hsa-miR-144 | 24 |
| hsa-miR-548a-5p | 24 |
| mmu-miR-464 | 24 |
| hsa-miR-101 | 23 |
| hsa-miR-151-5p | 23 |
| hsa-miR-181a | 23 |
| hsa-miR-200c | 23 |
| hsa-miR-548d-5p | 23 |
| mmu-miR-466b-3-3p | 23 |
| mmu-miR-666-3p | 23 |
| mmu-miR-383b-5p | 23 |
| hsa-let-7d* | 22 |
| hsa-miR-509-3-5p | 22 |
| hsa-miR-574-5p | 22 |
| mmu-miR-466f-3p | 22 |
| hsa-miR-130a | 21 |
| hsa-miR-130b | 21 |
| hsa-miR-421 | 21 |
| hsa-miR-548b-5p | 21 |
| hsa-miR-55 | 21 |
| mmu-miR-466d-5p | 21 |
| hsa-let-7b* | 20 |
| hsa-miR-238 | 20 |
| hsa-miR-34a | 20 |
| hsa-miR-518d-5p | 20 |
| mmu-miR-721 | 20 |
| mmu-miR-878-3p | 20 |
| hsa-let-7f-1* | 19 |
| hsa-miR-148a | 19 |
| hsa-miR-181c | 19 |
| hsa-miR-423-3p | 19 |
| hsa-miR-454 | 19 |
| hsa-miR-487a | 19 |
| hsa-miR-888 | 19 |
| mmu-miR-465a-5p | 19 |
| hsa-let-7a* | 18 |
| hsa-let-7f-2* | 18 |
| hsa-miR-199a-3p | 18 |
| hsa-miR-19a | 18 |
| hsa-miR-202 | 18 |
| hsa-miR-301a | 18 |
| hsa-miR-509-5p | 18 |
| hsa-miR-518a-3p | 18 |
| hsa-miR-768-5p | 18 |
| mmu-miR-466d-3p | 18 |
| mmu-miR-540-3p | 18 |
| mmu-miR-696 | 18 |
| hsa-let-7e* | 17 |
| hsa-miR-125a-3p | 17 |
| hsa-miR-135a* | 17 |
| hsa-miR-148b | 17 |
| hsa-miR-148b* | 17 |
| hsa-miR-154* | 17 |
| hsa-miR-15b | 17 |
| hsa-miR-190 | 17 |
| hsa-miR-210 | 17 |
| hsa-miR-212 | 17 |
| hsa-miR-296-5p | 17 |
| hsa-miR-369-3p | 17 |
| hsa-miR-485-3p | 17 |
| hsa-miR-487b | 17 |
| hsa-miR-489 | 17 |
| hsa-miR-509-3p | 17 |
| hsa-miR-518b | 17 |
| hsa-miR-518e | 17 |
| hsa-miR-518f | 17 |
| hsa-miR-548d-3p | 17 |
| hsa-miR-561 | 17 |
| hsa-miR-604 | 17 |
| hsa-miR-607 | 17 |
| hsa-miR-663 | 17 |
| hsa-miR-876-3p | 17 |
| hsa-miR-877* | 17 |
| mmu-miR-350 | 17 |
| mmu-miR-466a-3p | 17 |
| mmu-miR-466f-5p | 17 |
| mmu-miR-547 | 17 |
| hsa-let-7c* | 16 |
| hsa-miR-136* | 16 |
| hsa-miR-138 | 16 |
| hsa-miR-302a* | 16 |
| hsa-miR-330-5p | 16 |
| hsa-miR-358-5p | 16 |
| hsa-miR-376a | 16 |
| hsa-miR-376b | 16 |
| hsa-miR-376c | 16 |
| hsa-miR-377 | 16 |
| hsa-miR-450a | 16 |
| hsa-miR-493* | 16 |
| hsa-miR-516a-3p | 16 |
| hsa-miR-518c | 16 |
| hsa-miR-563 | 16 |
| hsa-miR-636 | 16 |
| hsa-miR-871 | 16 |
| mmu-miR-693-3p | 16 |
| mmu-miR-883b-3p | 16 |
| hsa-miR-132 | 15 |
| hsa-miR-133a | 15 |
| hsa-miR-137 | 15 |
| hsa-miR-143 | 15 |
| hsa-miR-148a* | 15 |
| hsa-miR-19b-1* | 15 |
| hsa-miR-200a | 15 |
| hsa-miR-20a* | 15 |
| hsa-miR-30b* | 15 |
| hsa-miR-326 | 15 |
| hsa-miR-33a* | 15 |
| hsa-miR-365 | 15 |
| hsa-miR-379 | 15 |
| hsa-miR-448 | 15 |
| hsa-miR-501-5p | 15 |
| hsa-miR-518d-3p | 15 |
| hsa-miR-520c-3p | 15 |
| hsa-miR-542-3p | 15 |
| hsa-miR-554 | 15 |
| hsa-miR-560 | 15 |
| hsa-miR-570 | 15 |
| hsa-miR-572 | 15 |
| hsa-miR-588 | 15 |
| hsa-miR-615-5p | 15 |
| hsa-miR-616* | 15 |
| hsa-miR-620 | 15 |
| hsa-miR-651 | 15 |
| hsa-miR-672 | 15 |
| hsa-miR-765 | 15 |
| hsa-miR-10b* | 14 |
| hsa-miR-130a* | 14 |
| hsa-miR-133b | 14 |
| hsa-miR-146b-3p | 14 |
| hsa-miR-149 | 14 |
| hsa-miR-16 | 14 |
| hsa-miR-190b | 14 |
| hsa-miR-198 | 14 |
| hsa-miR-199b-5p | 14 |
| hsa-miR-19a* | 14 |
| hsa-miR-19b | 14 |
| hsa-miR-19b-2* | 14 |
| hsa-miR-216b | 14 |
| hsa-miR-34c-5p | 14 |
| hsa-miR-374b | 14 |
| hsa-miR-423-5p | 14 |
| hsa-miR-431* | 14 |
| hsa-miR-503 | 14 |
| hsa-miR-517c | 14 |
| hsa-miR-518e* | 14 |
| hsa-miR-520d-3p | 14 |
| hsa-miR-520d-5p | 14 |
| hsa-miR-520e | 14 |
| hsa-miR-523 | 14 |
| hsa-miR-543 | 14 |
| hsa-miR-576-5p | 14 |
| hsa-miR-586 | 14 |
| hsa-miR-587 | 14 |
| hsa-miR-598 | 14 |
| hsa-miR-644 | 14 |
| hsa-miR-671-5p | 14 |
| hsa-miR-744 | 14 |
| hsa-miR-802 | 14 |
| hsa-miR-922 | 14 |
| hsa-miR-923 | 14 |
| mmu-miR-467b | 14 |
| mmu-miR-471 | 14 |
| mmu-miR-545 | 14 |
| mmu-miR-686 | 14 |
| mmu-miR-764-3p | 14 |
| mo-miR-352 | 14 |
| mo-miR-564 | 14 |
| hsa-let-7e | 13 |
| hsa-let-7l | 13 |
| hsa-let-7l* | 13 |
| hsa-miR-10b | 13 |
| hsa-miR-122 | 13 |
| hsa-miR-142-5p | 13 |
| hsa-miR-147 | 13 |
| hsa-miR-147b | 13 |
| hsa-miR-152 | 13 |
| hsa-miR-155 | 13 |
| hsa-miR-15a | 13 |
| hsa-miR-182 | 13 |
| hsa-miR-195 | 13 |
| hsa-miR-195b | 13 |
| hsa-miR-208b | 13 |
| hsa-miR-22 | 13 |
| hsa-miR-224 | 13 |
| hsa-miR-28-5p | 13 |
| hsa-miR-299-5p | 13 |
| hsa-miR-29b | 13 |
| hsa-miR-301b | 13 |
| hsa-miR-30a* | 13 |
| hsa-miR-330-3p | 13 |
| hsa-miR-331-3p | 13 |
| hsa-miR-33b | 13 |
| hsa-miR-369-5p | 13 |
| hsa-miR-374a | 13 |
| hsa-miR-377* | 13 |
| hsa-miR-455-3p | 13 |
| hsa-miR-490-3p | 13 |
| hsa-miR-517b | 13 |
| hsa-miR-518f* | 13 |
| hsa-miR-519a | 13 |
| hsa-miR-520a-3p | 13 |
| hsa-miR-520b | 13 |
| hsa-miR-548a-3p | 13 |
| hsa-miR-557 | 13 |
| hsa-miR-573 | 13 |
| hsa-miR-579 | 13 |
| hsa-miR-609 | 13 |
| hsa-miR-626 | 13 |
| hsa-miR-647 | 13 |
| hsa-miR-653 | 13 |
| hsa-miR-770-5p | 13 |
| hsa-miR-921 | 13 |
| hsa-miR-934 | 13 |
| hsa-miR-944 | 13 |
| hsa-miR-96 | 13 |
| mmu-miR-290-3p | 13 |
| mmu-miR-344 | 13 |
| mmu-miR-465c-5p | 13 |
| mmu-miR-466a-5p | 13 |
| mmu-miR-466g | 13 |
| mmu-miR-467d | 13 |
| mmu-miR-659a | 13 |
| mmu-miR-684 | 13 |
| mmu-miR-689 | 13 |
| mmu-miR-805 | 13 |
| gga-miR-460 | 12 |
| hsa-let-7b | 12 |
| hsa-miR-126* | 12 |
| hsa-miR-141* | 12 |
| hsa-miR-149* | 12 |
| hsa-miR-188-5p | 12 |
| hsa-miR-191 | 12 |
| hsa-miR-196a | 12 |
| hsa-miR-217 | 12 |
| hsa-miR-219-2-3p | 12 |
| hsa-miR-220c | 12 |
| hsa-miR-222 | 12 |
| hsa-miR-27b | 12 |
| hsa-miR-28-3p | 12 |
| hsa-miR-296-3p | 12 |
| hsa-miR-29a | 12 |
| hsa-miR-30c-2* | 12 |
| hsa-miR-34b | 12 |
| hsa-miR-362-3p | 12 |
| hsa-miR-382 | 12 |
| hsa-miR-450b-5p | 12 |
| hsa-miR-451 | 12 |
| hsa-miR-492 | 12 |
| hsa-miR-498 | 12 |
| hsa-miR-519c-3p | 12 |
| hsa-miR-519d | 12 |
| hsa-miR-520h | 12 |
| hsa-miR-542-5p | 12 |
| hsa-miR-548c-3p | 12 |
| hsa-miR-564 | 12 |
| hsa-miR-578 | 12 |
| hsa-miR-590-5p | 12 |
| hsa-miR-612 | 12 |
| hsa-miR-624 | 12 |
| hsa-miR-638 | 12 |
| hsa-miR-648 | 12 |
| hsa-miR-655 | 12 |
| hsa-miR-877 | 12 |
| hsa-miR-891a | 12 |
| hsa-miR-891b | 12 |
| hsa-miR-892a | 12 |
| hsa-miR-93 | 12 |
| hsa-miR-943 | 12 |
| hsa-miR-98 | 12 |
| hsa-miR-59b* | 12 |
| mmu-miR-291b-3p | 12 |
| mmu-miR-322 | 12 |
| mmu-miR-351 | 12 |
| mmu-miR-463 | 12 |
| mmu-miR-465b-5p | 12 |
| mmu-miR-466h | 12 |
| mmu-miR-467e | 12 |
| mmu-miR-682 | 12 |
| mmu-miR-595 | 12 |
| mmu-miR-703 | 12 |
| mmu-miR-741 | 12 |
| mmu-miR-743a | 12 |
| mmu-miR-763 | 12 |
| mo-miR-333 | 12 |
| mo-miR-336 | 12 |
| hsa-let-7a | 11 |
| hsa-let-7d | 11 |
| hsa-let-7g | 11 |
| hsa-miR-106b | 11 |
| hsa-miR-107 | 11 |
| hsa-miR-124 | 11 |
| hsa-miR-139-3p | 11 |
| hsa-miR-146a* | 11 |
| hsa-miR-150 | 11 |
| hsa-miR-185 | 11 |
| hsa-miR-186* | 11 |
| hsa-miR-187 | 11 |
| hsa-miR-18a | 11 |
| hsa-miR-192 | 11 |
| hsa-miR-193a-3p | 11 |
| hsa-miR-199a-5p | 11 |
| hsa-miR-200b* | 11 |
| hsa-miR-205 | 11 |
| hsa-miR-208 | 11 |
| hsa-miR-23a | 11 |
| hsa-miR-23b | 11 |
| hsa-miR-27a | 11 |
| hsa-miR-297 | 11 |
| hsa-miR-29c | 11 |
| hsa-miR-34c-3p | 11 |
| hsa-miR-381 | 11 |
| hsa-miR-483-5p | 11 |
| hsa-miR-497 | 11 |
| hsa-miR-499-5p | 11 |
| hsa-miR-513-5p | 11 |
| hsa-miR-516b | 11 |
| hsa-miR-518a-5p | 11 |
| hsa-miR-519b-3p | 11 |
| hsa-miR-520a-5p | 11 |
| hsa-miR-520f | 11 |
| hsa-miR-521 | 11 |
| hsa-miR-522 | 11 |
| hsa-miR-524-5p | 11 |
| hsa-miR-568 | 11 |
| hsa-miR-576-3p | 11 |
| hsa-miR-580 | 11 |
| hsa-miR-608 | 11 |
| hsa-miR-621 | 11 |
| hsa-miR-656 | 11 |
| hsa-miR-661 | 11 |
| hsa-miR-886-5p | 11 |
| hsa-miR-92a-2* | 11 |
| hsa-miR-936 | 11 |
| mmu-miR-294 | 11 |
| mmu-miR-341 | 11 |
| mmu-miR-467c | 11 |
| mmu-miR-470 | 11 |
| mmu-miR-670 | 11 |
| mmu-miR-673-3p | 11 |
| mmu-miR-691 | 11 |
| mmu-miR-692 | 11 |
| mmu-miR-707 | 11 |
| mmu-miR-710 | 11 |
| mmu-miR-883a-5p | 11 |
| hsa-let-7c | 10 |
| hsa-miR-1 | 10 |
| hsa-miR-103 | 10 |
| hsa-miR-125a-5p | 10 |
| hsa-miR-129-5p | 10 |
| hsa-miR-135a | 10 |
| hsa-miR-140-3p | 10 |
| hsa-miR-146a | 10 |
| hsa-miR-146b-5p | 10 |
| hsa-miR-151-3p | 10 |
| hsa-miR-16-2* | 10 |
| hsa-miR-17 | 10 |
| hsa-miR-183 | 10 |
| hsa-miR-18b | 10 |
| hsa-miR-191* | 10 |
| hsa-miR-193a-5p | 10 |
| hsa-miR-193b | 10 |
| hsa-miR-20b | 10 |
| hsa-miR-221* | 10 |
| hsa-miR-223* | 10 |
| hsa-miR-300 | 10 |
| hsa-miR-30c-1* | 10 |
| hsa-miR-30d* | 10 |
| hsa-miR-338-3p | 10 |
| hsa-miR-33a | 10 |
| hsa-miR-342-3p | 10 |
| hsa-miR-34a* | 10 |
| hsa-miR-363* | 10 |
| hsa-miR-374b* | 10 |
| hsa-miR-357 | 10 |
| hsa-miR-376a* | 10 |
| hsa-miR-383 | 10 |
| hsa-miR-409-5p | 10 |
| hsa-miR-449b | 10 |
| hsa-miR-452 | 10 |
| hsa-miR-491-5p | 10 |
| hsa-miR-494 | 10 |
| hsa-miR-508-5p | 10 |
| hsa-miR-515-5p | 10 |
| hsa-miR-517a | 10 |
| hsa-miR-518c* | 10 |
| hsa-miR-525-5p | 10 |
| hsa-miR-548b-3p | 10 |
| hsa-miR-549 | 10 |
| hsa-miR-550* | 10 |
| hsa-miR-553 | 10 |
| hsa-miR-559 | 10 |
| hsa-miR-562 | 10 |
| hsa-miR-571 | 10 |
| hsa-miR-575 | 10 |
| hsa-miR-582-3p | 10 |
| hsa-miR-602 | 10 |
| hsa-miR-640 | 10 |
| hsa-miR-641 | 10 |
| hsa-miR-760 | 10 |
| hsa-miR-767-3p | 10 |
| hsa-miR-767-5p | 10 |
| hsa-miR-801 | 10 |
| hsa-miR-889 | 10 |
| hsa-miR-892b | 10 |
| hsa-miR-9 | 10 |
| hsa-miR-92a-1* | 10 |
| hsa-miR-92b | 10 |
| hsa-miR-99a* | 10 |
| hsa-miR-99b | 10 |
| mmu-miR-434-5p | 10 |
| mmu-miR-466b-5p | 10 |
| mmu-miR-466c-5p | 10 |
| mmu-miR-466e-5p | 10 |
| mmu-miR-468 | 10 |
| mmu-miR-667 | 10 |
| mmu-miR-680 | 10 |
| mmu-miR-681 | 10 |
| mmu-miR-593-5p | 10 |
| mmu-miR-701 | 10 |
| mmu-miR-702 | 10 |
| mmu-miR-705 | 10 |
| mmu-miR-713 | 10 |
| mmu-miR-742 | 10 |
| mmu-miR-764-5p | 10 |
| mmu-miR-883a-3p | 10 |
| mo-miR-349 | 10 |
| gga-miR-757 | 9 |
| hsa-miR-105 | 9 |
| hsa-miR-122* | 9 |
| hsa-miR-124* | 9 |
| hsa-miR-125b | 9 |
| hsa-miR-125b-1* | 9 |
| hsa-miR-128a | 9 |
| hsa-miR-132* | 9 |
| hsa-miR-138-2* | 9 |
| hsa-miR-141 | 9 |
| hsa-miR-142-3p | 9 |
| hsa-miR-143* | 9 |
| hsa-miR-150* | 9 |
| hsa-miR-15a* | 9 |
| hsa-miR-16-1* | 9 |
| hsa-miR-17* | 9 |
| hsa-miR-194 | 9 |
| hsa-miR-204 | 9 |
| hsa-miR-220b | 9 |
| hsa-miR-221 | 9 |
| hsa-miR-222* | 9 |
| hsa-miR-24 | 9 |
| hsa-miR-298 | 9 |
| hsa-miR-29b-1* | 9 |
| hsa-miR-302a | 9 |
| hsa-miR-302b | 9 |
| hsa-miR-30c | 9 |
| hsa-miR-30e* | 9 |
| hsa-miR-323-5p | 9 |
| hsa-miR-328 | 9 |
| hsa-miR-342-5p | 9 |
| hsa-miR-371-3p | 9 |
| hsa-miR-411 | 9 |
| hsa-miR-422a | 9 |
| hsa-miR-424 | 9 |
| hsa-miR-431 | 9 |
| hsa-miR-432 | 9 |
| hsa-miR-452* | 9 |
| hsa-miR-455-5p | 9 |
| hsa-miR-493 | 9 |
| hsa-miR-495 | 9 |
| hsa-miR-507 | 9 |
| hsa-miR-512-3p | 9 |
| hsa-miR-513-3p | 9 |
| hsa-miR-517* | 9 |
| hsa-miR-520g | 9 |
| hsa-miR-541 | 9 |
| hsa-miR-555 | 9 |
| hsa-miR-556-3p | 9 |
| hsa-miR-574-3p | 9 |
| hsa-miR-582-5p | 9 |
| hsa-miR-589 | 9 |
| hsa-miR-591 | 9 |
| hsa-miR-595 | 9 |
| hsa-miR-601 | 9 |
| hsa-miR-603 | 9 |
| hsa-miR-631 | 9 |
| hsa-miR-671-3p | 9 |
| hsa-miR-675 | 9 |
| hsa-miR-7 | 9 |
| hsa-miR-769-3p | 9 |
| hsa-miR-872 | 9 |
| hsa-miR-873 | 9 |
| hsa-miR-886-3p | 9 |
| hsa-miR-9* | 9 |
| hsa-miR-92a | 9 |
| hsa-miR-940 | 9 |
| hsa-miR-99a | 9 |
| mmu-miR-291a-3p | 9 |
| mmu-miR-327 | 9 |
| mmu-miR-467a | 9 |
| mmu-miR-669b | 9 |
| mmu-miR-678 | 9 |
| mmu-miR-683 | 9 |
| mmu-miR-690 | 9 |
| mmu-miR-717 | 9 |
| hsa-let-7g* | 8 |
| hsa-miR-106b* | 8 |
| hsa-miR-126 | 8 |
| hsa-miR-271-5p | 8 |
| hsa-miR-138-1* | 8 |
| hsa-miR-153 | 8 |
| hsa-miR-181c* | 8 |
| hsa-miR-187* | 8 |
| hsa-miR-200a* | 8 |
| hsa-miR-20a | 8 |
| hsa-miR-211 | 8 |
| hsa-miR-214 | 8 |
| hsa-miR-215 | 8 |
| hsa-miR-218-1* | 8 |
| hsa-miR-223 | 8 |
| hsa-miR-25 | 8 |
| hsa-miR-299-3p | 8 |
| hsa-miR-29a* | 8 |
| hsa-miR-29c* | 8 |
| hsa-miR-329 | 8 |
| hsa-miR-337-3p | 8 |
| hsa-miR-339-3p | 8 |
| hsa-miR-367* | 8 |
| hsa-miR-379* | 8 |
| hsa-miR-380* | 8 |
| hsa-miR-410 | 8 |
| hsa-miR-429 | 8 |
| hsa-miR-449a | 8 |
| hsa-miR-450b-3p | 8 |
| hsa-miR-486-3p | 8 |
| hsa-miR-497* | 8 |
| hsa-miR-506 | 8 |
| hsa-miR-508-3p | 8 |
| hsa-miR-539 | 8 |
| hsa-miR-545 | 8 |
| hsa-miR-545* | 8 |
| hsa-miR-565 | 8 |
| hsa-miR-569 | 8 |
| hsa-miR-593* | 8 |
| hsa-miR-613 | 8 |
| hsa-miR-615-5p | 8 |
| hsa-miR-625 | 8 |
| hsa-miR-628-5p | 8 |
| hsa-miR-639 | 8 |
| hsa-miR-642 | 8 |
| hsa-miR-643 | 8 |
| hsa-miR-645 | 8 |
| hsa-miR-645-5p | 8 |
| hsa-miR-766 | 8 |
| hsa-miR-768-3p | 8 |
| hsa-miR-885-3p | 8 |
| hsa-miR-92b* | 8 |
| hsa-miR-937 | 8 |
| hsa-miR-938 | 8 |
| hsa-miR-941 | 8 |
| mmi-miR-189 | 8 |
| mmu-miR-201 | 8 |
| mmu-miR-292-3p | 8 |
| mmu-miR-434-3p | 8 |
| mmu-miR-673-5p | 8 |
| mmu-miR-697 | 8 |
| mmu-miR-699 | 8 |
| mmu-miR-743b-3p | 8 |
| mmu-miR-743b-5p | 8 |
| mmu-miR-761 | 8 |
| mmu-miR-880 | 8 |
| mmu-miR-882 | 8 |
| hsa-let-7f | 7 |
| hsa-miR-101* | 7 |
| hsa-miR-106a | 7 |
| hsa-miR-106a* | 7 |
| hsa-miR-10a | 7 |
| hsa-miR-125b-2* | 7 |
| hsa-miR-129-3p | 7 |
| hsa-miR-135b | 7 |
| hsa-miR-139-5p | 7 |
| hsa-miR-140-5p | 7 |
| hsa-miR-15b* | 7 |
| hsa-miR-181a* | 7 |
| hsa-miR-182* | 7 |
| hsa-miR-183* | 7 |
| hsa-miR-188-3p | 7 |
| hsa-miR-195* | 7 |
| hsa-miR-203 | 7 |
| hsa-miR-206 | 7 |
| hsa-miR-21 | 7 |
| hsa-miR-219-1-3p | 7 |
| hsa-miR-219-5p | 7 |
| hsa-miR-220 | 7 |
| hsa-miR-23b* | 7 |
| hsa-miR-26a | 7 |
| hsa-miR-26a-2* | 7 |
| hsa-miR-29b-2* | 7 |
| hsa-miR-302c | 7 |
| hsa-miR-302c* | 7 |
| hsa-miR-30a | 7 |
| hsa-miR-30b | 7 |
| hsa-miR-31* | 7 |
| hsa-miR-324-5p | 7 |
| hsa-miR-325 | 7 |
| hsa-miR-335* | 7 |
| hsa-miR-337-5p | 7 |
| hsa-miR-340 | 7 |
| hsa-miR-340* | 7 |
| hsa-miR-362-5p | 7 |
| hsa-miR-367 | 7 |
| hsa-miR-384 | 7 |
| hsa-miR-424* | 7 |
| hsa-miR-432* | 7 |
| hsa-miR-454* | 7 |
| hsa-miR-484 | 7 |
| hsa-miR-490-5p | 7 |
| hsa-miR-491-3p | 7 |
| hsa-miR-500 | 7 |
| hsa-miR-502-5p | 7 |
| hsa-miR-505 | 7 |
| hsa-miR-514 | 7 |
| hsa-miR-515-3p | 7 |
| hsa-miR-519e* | 7 |
| hsa-miR-544 | 7 |
| hsa-miR-556-5p | 7 |
| hsa-miR-567 | 7 |
| hsa-miR-577 | 7 |
| hsa-miR-590-3p | 7 |
| hsa-miR-596 | 7 |
| hsa-miR-600 | 7 |
| hsa-miR-605 | 7 |
| hsa-miR-616 | 7 |
| hsa-miR-619 | 7 |
| hsa-miR-632 | 7 |
| hsa-miR-633 | 7 |
| hsa-miR-649 | 7 |
| hsa-miR-665 | 7 |
| hsa-miR-668 | 7 |
| hsa-miR-674 | 7 |
| hsa-miR-769-5p | 7 |
| hsa-miR-887 | 7 |
| hsa-miR-93* | 7 |
| mmu-miR-290-5p | 7 |
| mmu-miR-291b-5p | 7 |
| mmu-miR-295 | 7 |
| mmu-miR-343 | 7 |
| mmu-miR-685 | 7 |
| mmu-miR-687 | 7 |
| mmu-miR-804 | 7 |
| mmu-miR-881 | 7 |
| hsa-miR-10a* | 6 |
| hsa-miR-129* | 6 |
| hsa-miR-136 | 6 |
| hsa-miR-145 | 6 |
| hsa-miR-181a-2* | 6 |
| hsa-miR-185* | 6 |
| hsa-miR-186 | 6 |
| hsa-miR-194* | 6 |
| hsa-miR-202* | 6 |
| hsa-miR-21* | 6 |
| hsa-miR-22* | 6 |
| hsa-miR-26b | 6 |
| hsa-miR-26b* | 6 |
| hsa-miR-27a* | 6 |
| hsa-miR-302d | 6 |
| hsa-miR-302d* | 6 |
| hsa-miR-30e | 6 |
| hsa-miR-324-3p | 6 |
| hsa-miR-335 | 6 |
| hsa-miR-34b* | 6 |
| hsa-miR-361-3p | 6 |
| hsa-miR-363 | 6 |
| hsa-miR-370 | 6 |
| hsa-miR-380 | 6 |
| hsa-miR-411* | 6 |
| hsa-miR-412 | 6 |
| hsa-miR-425* | 6 |
| hsa-miR-483-3p | 6 |
| hsa-miR-486-5p | 6 |
| hsa-miR-488 | 6 |
| hsa-miR-488* | 6 |
| hsa-miR-496 | 6 |
| hsa-miR-499-3p | 6 |
| hsa-miR-502-3p | 6 |
| hsa-miR-532-5p | 6 |
| hsa-miR-550 | 6 |
| hsa-miR-558 | 6 |
| hsa-miR-585 | 6 |
| hsa-miR-592 | 6 |
| hsa-miR-593 | 6 |
| hsa-miR-597 | 6 |
| hsa-miR-611 | 6 |
| hsa-miR-614 | 6 |
| hsa-miR-624* | 6 |
| hsa-miR-629 | 6 |
| hsa-miR-629* | 6 |
| hsa-miR-634 | 6 |
| hsa-miR-654-3p | 6 |
| hsa-miR-708 | 6 |
| hsa-miR-7-2* | 6 |
| hsa-miR-758 | 6 |
| hsa-miR-875-3p | 6 |
| hsa-miR-875-5p | 6 |
| hsa-miR-920 | 6 |
| hsa-miR-939 | 6 |
| mmu-miR-291a-5p | 6 |
| mmu-miR-292-5p | 6 |
| mmu-miR-293 | 6 |
| mmu-miR-667 | 6 |
| mmu-miR-669c | 6 |
| mmu-miR-676 | 6 |
| mmu-miR-679 | 6 |
| mmu-miR-698 | 6 |
| mmu-miR-704 | 6 |
| mmu-miR-706 | 6 |
| mmu-miR-879 | 6 |
| hsa-miR-127-3p | 5 |
| hsa-miR-130b* | 5 |
| hsa-miR-192* | 5 |
| hsa-miR-193b* | 5 |
| hsa-miR-196a* | 5 |
| hsa-miR-197 | 5 |
| hsa-miR-216a | 5 |
| hsa-miR-27b* | 5 |
| hsa-miR-302b* | 5 |
| hsa-miR-31 | 5 |
| hsa-miR-32 | 5 |
| hsa-miR-331-5p | 5 |
| hsa-miR-371-5p | 5 |
| hsa-miR-373 | 5 |
| hsa-miR-378 | 5 |
| hsa-miR-378* | 5 |
| hsa-miR-485-5p | 5 |
| hsa-miR-500* | 5 |
| hsa-miR-501-3p | 5 |
| hsa-miR-512-5p | 5 |
| hsa-miR-524-3p | 5 |
| hsa-miR-526b* | 5 |
| hsa-miR-532-3p | 5 |
| hsa-miR-551b | 5 |
| hsa-miR-552 | 5 |
| hsa-miR-584 | 5 |
| hsa-miR-606 | 5 |
| hsa-miR-617 | 5 |
| hsa-miR-618 | 5 |
| hsa-miR-622 | 5 |
| hsa-miR-637 | 5 |
| hsa-miR-650 | 5 |
| hsa-miR-658 | 5 |
| hsa-miR-744* | 5 |
| hsa-miR-876-5p | 5 |
| hsa-miR-888* | 5 |
| hsa-miR-933 | 5 |
| mmu-miR-469 | 5 |
| mmu-miR-694 | 5 |
| mmu-miR-711 | 5 |
| mmu-miR-712 | 5 |
| mmu-miR-715 | 5 |
| mmu-miR-718 | 5 |
| mmu-miR-762 | 5 |
| mo-miR-347 | 5 |
| hsa-miR-100 | 4 |
| hsa-miR-100* | 4 |
| hsa-miR-134 | 4 |
| hsa-miR-135b* | 4 |
| hsa-miR-154 | 4 |
| hsa-miR-184 | 4 |
| hsa-miR-18b* | 4 |
| hsa-miR-218-2* | 4 |
| hsa-miR-23a* | 4 |
| hsa-miR-24-2* | 4 |
| hsa-miR-26a-1* | 4 |
| hsa-miR-32* | 4 |
| hsa-miR-339-5p | 4 |
| hsa-miR-33b* | 4 |
| hsa-miR-345 | 4 |
| hsa-miR-374a* | 4 |
| hsa-miR-409-3p | 4 |
| hsa-miR-433 | 4 |
| hsa-miR-510 | 4 |
| hsa-miR-516a-5p | 4 |
| hsa-miR-519e | 4 |
| hsa-miR-525-3p | 4 |
| hsa-miR-526b | 4 |
| hsa-miR-551a | 4 |
| hsa-miR-551b* | 4 |
| hsa-miR-583 | 4 |
| hsa-miR-628-3p | 4 |
| hsa-miR-630 | 4 |
| hsa-miR-635 | 4 |
| hsa-miR-646 | 4 |
| hsa-miR-662 | 4 |
| hsa-miR-708* | 4 |
| hsa-miR-874 | 4 |
| hsa-miR-885-5p | 4 |
| hsa-miR-935 | 4 |
| hsa-miR-942 | 4 |
| mmu-miR-207 | 4 |
| mmu-miR-540-5p | 4 |
| mmu-miR-666-5p | 4 |
| mmu-miR-688 | 4 |
| mmu-miR-720 | 4 |
| hsa-miR-105* | 3 |
| hsa-miR-144* | 3 |
| hsa-miR-155* | 3 |
| hsa-miR-18a* | 3 |
| hsa-miR-24-1* | 3 |
| hsa-miR-320 | 3 |
| hsa-miR-346 | 3 |
| hsa-miR-372 | 3 |
| hsa-miR-425 | 3 |
| hsa-miR-453 | 3 |
| hsa-miR-504 | 3 |
| hsa-miR-589* | 3 |
| hsa-miR-610 | 3 |
| hsa-miR-623 | 3 |
| hsa-miR-625* | 3 |
| hsa-miR-627 | 3 |
| hsa-miR-657 | 3 |
| hsa-miR-659 | 3 |
| hsa-miR-660 | 3 |
| hsa-miR-7-1* | 3 |
| hsa-miR-890 | 3 |
| hsa-miR-96* | 3 |
| mmu-miR-709 | 3 |
| gga-miR-456 | 2 |
| hsa-miR-214* | 2 |
| hsa-miR-25* | 2 |
| hsa-miR-30d | 2 |
| hsa-miR-323-3p | 2 |
| hsa-miR-361-5p | 2 |
| hsa-miR-505* | 2 |
| hsa-miR-511 | 2 |
| hsa-miR-541* | 2 |
| hsa-miR-566 | 2 |
| hsa-miR-599 | 2 |
| hsa-miR-924 | 2 |
| mmu-miR-465a-3p | 2 |
| mmu-miR-700 | 2 |
| mmu-miR-714 | 2 |
| mmu-miR-719 | 2 |
| mmu-miR-759 | 2 |
| mmu-miR-878-5p | 2 |
| hsa-miR-145* | 1 |
| hsa-miR-200c* | 1 |
| hsa-miR-373* | 1 |
| hsa-miR-581 | 1 |
| hsa-miR-652 | 1 |

TABLE 3
Functional annotation categories over-represented in genes
that are upregulated or down-regulated during skin aging

| | |
|---|---|
| upregulated in aged skin | downregulated in aged skin |

Over-represented Gene Ontology Biological Processes in genes

| | |
|---|---|
| response to wounding | anatomical structure development |
| defense response | system development |
| response to external stimulus | organ development |
| inflammatory response | multicellular organismal development |
| response to stress | developmental process |
| anatomical structure development | transcription front RNA polymerase II promoter |
| developmental process | regulation of transcription from RNA polymerase II promoter |
| organ development | |
| multicellular organismal process | |
| multicellular organismal development | |
| immune response | |
| regulation of biological quality | |
| response to other organism | |
| response to biotic stimulus | |
| cell-cell signaling | |
| immune system process | |
| multi-organism process | |
| cell proliferation | |
| response to stimulus | |
| system development | |
| negative regulation of biological process | |
| response to chemical stimulus | |
| taxis | |
| chemotaxis | |
| cell differentiation | |
| cellular developmental process | |
| ectoderm development | |
| negative regulation of apoptosis | |
| negative regulation of programmed cell death | |
| anatomical structure morphogenesis | |

Over-represented Gene Ontology Cellular Locations in genes

| | |
|---|---|
| extracellular region | extracellular region part |
| extracellular space | extracellular region |
| extracellular region part | proteinaceous extracellular matrix |
| | extracellular matrix |

Over-represented Gene Ontology Molecular Functions in genes

| | |
|---|---|
| endopeptidase activity | protein binding |
| protein binding | |
| peptidase activity | |
| plasminogen activator activity | |
| chemokine activity | |
| chemokine receptor binding | |
| serine-type endopeptidase activity | |
| cytokine activity | |
| serine-type peptidase activity | |
| serine hydrolase activity | |
| pattern binding | |
| receptor bidding | |
| G-protein-coupled receptor binding | |

Over-represented InterPro Domains in genes

Small chemokine, interleukin-8-like

Over-represented Pfam protein families in genes

ILB

Over-represented BIND protein categories m genes

transactivating regulatory protein

Over-represented Reactome protein categories in genes

plasminogen

Over-represented Genomic Association classes in genes

infection
immune
aging
unknown
neurological
cardiovascular
vision
normal variation
pharmacogenomic

Over-represented tissue categories in genes

Keratinocyte
Epidermis
Lung

1. A method for reversing signs of skin aging, the method comprising recalibrating the expression of two or more genes expressed in human skin tissue the expression of which has changed as a result of aging wherein the genes are selected form those genes listed in Table 1, the method comprising applying to the skin a compound that alters the expression of one or more genes selected from Table 1.

2. (canceled)

3. The method of claim 1 wherein the genes recalibrated comprise one or more genes involved in the biosynthesis or degradation of collagens and elastins, glycosaminoglycans or fibronectins.

4. (canceled)

5. The method of claim 4 wherein the compound comprises a chromatin remodeling protein or peptide.

6. The method of claim 1 wherein the compound alters the expression of one or more genes involved in the biosynthesis or degradation of collagens and elastins, glycosaminoglycans or fibronectins.

7. The method of claim 4 further comprising orally administering a compound having anti-aging properties wherein the compound applied to the skin and the orally administered compound provide a synergistic effects upon a change in expression of genes of Table 1.

8. (canceled)

9. (canceled)

10. The method of claim 1 comprising a method for amplifying expression of genes, which genes are normally activated by hormones, drugs, extracellular and other environmental stimuli, the method comprising administering to a subject a therapeutic amount of chromatin remodeling protein or peptide or a variant or derivative thereof, thereby increasing chromatin accessibility in the promoter region of the gene due to acetylation of lysine 16 at the amino-terminal ends of histone H4 of nucleosome.

11. The method of claim 10 wherein the peptide is lunasin or a variant or derivative thereof.

12. A composition for reducing the signs of aging of the skin, the composition comprising one or more substances that alter the expression of genes involved in the biosynthesis or degradation of a substance selected from the group consisting of collagens and elastins, glycosaminoglycans, proteoglycans, mucopolysaccharides, hyaluronic acid, fibronectins and extracellular matrix proteins in the dermis.

13. The composition of claim 12 wherein the composition comprises a chromatin remodeling protein or peptide or variant thereof.

14. The composition of claim 12 wherein the composition comprises lunasin.

15. The composition of claim 12 comprising one or more substances that increase the expression of genes involved in the biosynthesis of type I or type II collagen.

16. The composition of claim 12 comprising one or more substances that reduce the enzymatic activity of collagenase.

17. The composition of claim 12 comprising one or more substances that reduce the enzymatic activity of enzymes that degrade fibrin or glycosaminoglycans, or that increase the production or maintenance of glycosaminoglycans or proteoglycans.

18. The composition of claim 12 comprising one or more substances that reduce the enzymatic activity of MMP enzymes (matrix-metalloproteinases) that degrade collagen.

19. The composition of claim 12 comprising one or more substances that increase type I and III procollagen synthesis.

20. The composition of claim 12 comprising one or more substances that decrease production of MMPs.

21. The composition of claim 12 comprising one or more substances that decrease production of compounds selected from the group consisting of MMP-1, MMP-2 (gelatinase A), MMP-3, and MMP-9.

22. The composition of claim 12 comprising one or more substances that maintain or increase the number of fibroblasts present in the dermis and the number of collagen fibrils and elastin fibers in the dermis, or one or more substances that maintain or increase the 3-dimensional extracellular matrix structure of collagen, elastin, or other extracellular matrix proteins in the dermis.

23. (canceled)

24. The composition of claim 12 comprising a combination of phytochemicals, nutrients, minerals and vitamins.

25. A method for identifying new compounds that will alter aging-related gene expression profiles and signatures, the method comprising: (i) providing a skin tissue sample, (ii) measuring the expression of a selected plurality of aging-related genes selected from Table 1, (iii) exposing skin tissue to a test compound, (iv) re-measuring the expression of the genes, (v) identifying a compound that changes the expression of the selected plurality of genes wherein the change in expression is at least two-fold.
